(19) United States
(12) Patent Application Publication
(10) Pub. No.: US 2015/0125883 A1
(43) Pub. Date: May 7, 2015

(54) METHOD OF ISOLATING AND CHARACTERIZING MICROORGANISMS THAT ARE TARGETS OF HOST IMMUNE RESPONSES

(71) Applicant: Washington University, St. Louis, MO (US)

(72) Inventors: Jeffrey I. Gordon, St. Louis, MO (US); Andrew Kau, St. Louis, MO (US); Chyi-Song Hsieu, St. Louis, MO (US); Sindhuja Rao, St. Louis, MO (US); Nathan P. Mcnulty, St. Louis, MO (US)

(21) Appl. No.: 14/398,087

(22) PCT Filed: Apr. 30, 2013
(86) PCT No.: PCT/US13/38898

§ 371 Date: Oct. 30, 2014

Related U.S. Application Data
(60) Provisional application No. 61/640,362, filed on Apr. 30, 2012.

(51) Int. Cl. G01N 33/569; G01N 33/50

(52) U.S. Cl. CPC G01N 33/56916; G01N 33/56911; G01N 33/5023; G01N 33/5088; G01N 2500/10
USPC 435/7.32; 702/19

(57) ABSTRACT

The present invention encompasses methods of isolating, identifying, and characterizing microorganisms present in a microbial community occupying a body habitat/surface of a healthy or unhealthy human or animal. More particularly, the invention relates to methods of isolating and identifying viable microorganisms that interact with a host's immune system.

### CROSS-REFERENCE TO RELATED APPLICATIONS

This application claims the priority of PCT Application PCT/US2013/038898, filed Apr. 30, 2013, which claims the priority of U.S. provisional application No. 61/640,362, filed Apr. 30, 2012, each of which is hereby incorporated by reference in its entirety.

### GOVERNMENTAL RIGHTS

This invention was made with government support under F32 DK091044 awarded by the National Institute of Diabetes and Digestive and Kidney Diseases. The government has certain rights in the invention.

### FIELD OF THE INVENTION

The present invention relates to methods of isolating, identifying, and characterizing microorganisms present in a microbial community occupying a body habitat/surface of a healthy or unhealthy human or animal. More particularly, the invention relates to methods of isolating and identifying microorganisms that interact with a host's immune system.

### BACKGROUND OF THE INVENTION

The generation of an immune response to a particular microorganism may provide valuable information when linked to a particular physiological state. For example, in non-pathological states, bacteria that are targets of an immune response are probably those bacteria best adapted to survive in the host. In pathological states, disease causing microbes may displace the normal microbiota, becoming a new target of the immune system. It is not known in the art if and/or to what extent, microbial exposures, diet, and other factors provoke changes in the microbial community structure and/or the antigenic features of community members.

This is particularly true of mucosal barriers, where characterizing its function in health and disease is critical for understanding and modulating the interactions between indigenous microbial communities in various body habitats (microbiota) and the host's immune system. Given the complexity of microbiota, and its variation as a function of individual, age, physiologic state and lifestyle, defining which organisms evoke and which organisms modulate immune responses requires an unbiased tool for identifying such organisms, whether they be bacterial, archaeal or eukaryotic. The ability to identify as well as retrieve such organisms in a viable form for further characterization of their properties either in vitro or in vivo after transfer to other hosts, provides a way for identifying disease causing as well as beneficial disease modifying or health promoting organisms (e.g. new probiotics). IgA is a major component of the mucosal immune response that aids in protecting and maintaining barrier function at mucosal surfaces. As a component of the adaptive immune response, IgA is produced by B cell/plasma cells that reside in mucosal surfaces, and is actively transported across mucosal epithelial surfaces into the sinuses, airways, and, in particular, into the lumen of the gastrointestinal tract where an estimated eight grams of IgA is produced by an individual on a daily basis. IgA functions by binding bacterial, food and other antigens to sequester them away from the mucosal surface and prevent direct interaction with the host, a principle known as “immune exclusion”.

The ability to identify consortia of bacteria known by the immune system at any given point in time may have important prognostic and therapeutic implications. Thus, there is a need in the art for methods to isolate, identify and characterize viable microorganisms that are targeted by the immune system, as well as additional methods to apply these new tools to screen for and select therapeutic interventions.

### SUMMARY OF THE INVENTION

One aspect of the invention encompasses a method for identifying a physiological state of a subject. The method comprises: (a) combining one or more biological samples comprising an immune system: microorganism complex obtained from the subject with one or more detection agents; (b) sorting, in vitro, the one or more samples into two populations: a detection agent bound immune system: microorganism complex population and an unbound immune system: microorganism complex population; (c) identifying the taxonomic composition of one or more detection agent bound immune system: microorganism complex populations and identifying the taxonomic composition of one or more unbound immune system: microorganism complex populations from the one or more samples; (d) calculating a strength of enrichment for an identified taxon in the detection agent bound population compared to the unbound population from each sample, wherein a strength of enrichment value greater than zero indicates enrichment in the detection agent bound population; and (e) identifying the physiological state of the subject by comparing the taxa enriched in the detection agent bound population of the subject to one or more reference samples each associated with a physiological state, wherein if the enriched taxa are similar between the subject and the reference sample, the subject has the physiological state associated with the reference sample. In some embodiments, the physiological state is proper functioning of the mucosal barrier including its immune cell population, and the disruption of that function, as for example in the case of forms of malnutrition, where the subject is a mammal, the biological sample is a fecal sample, and the detection agent is an anti-IgA antibody. The invention may further comprise use of a compound, a biologic, a probioitic, a prebiotic, a synbiotic, an antibiotic, a change in diet, or a combination thereof, in the treatment of a subject in a physiological state identified by the methods of the invention. The invention may also further comprise the step of administering to the subject a compound, a biologic, a probioitic, a prebiotic, a synbiotic, an antibiotic, a change in diet, or a combination thereof based on the identified physiological state of the subject.

Another aspect of the invention encompasses a method of identifying one or more taxa targeted by the immune system of a subject. The method comprises: (a) mixing a biological sample comprising microorganisms from different taxa from the subject with one or more detection agents; (b) sorting the sample into two populations: a detection agent bound microorganism population and an unbound microorganism population; (c) identifying the taxonomic composition of the detection agent bound microorganism population and the unbound microorganism population; (d) comparing the taxonomic composition of the detection agent bound microorganism population to the unbound microorganism population; and (e) calculating a strength of enrichment for each taxon in the detection agent bound population; wherein a strength of enrichment value greater than zero indicates enrichment of the identified taxa in the detection agent bound population and targeting by the immune system. In some embodiments, the biological sample comprises at least one immunoglobulin: microorganism complex, and, the detection agent is specific for an immunoglobulin (Ig) selected from the group consisting of IgG, IgM, IgE, IgA, IgD, and mixtures thereof. The methods of the invention may further comprise culturing the detection agent bound microorganism population, wherein the method of culturing is selected from the group consisting of (i) inoculating the detection agent bound microorganism population into a germ free animal, (ii) growing the detection agent bound microorganism population in vitro using standard anaerobic techniques, and (iii) a combination thereof. The methods of the invention may also further comprise the step of administering to the subject a compound, a biologic, a probioitic, a prebiotic, a synbiotic, an antibiotic, a change in diet, or a combination thereof comprising the microorganisms present in one or more identified taxa. The methods of the invention may also further comprise use of a compound, a biologic, a probioitic, a prebiotic, a synbiotic, an antibiotic, a change in diet, or a combination thereof, comprising the microorganisms present in one or more taxa identified by the methods of the invention in the modulation of the immune system of the subject.

Another aspect of the invention encompasses a method of screening for a therapeutic intervention effective at modulating the immune response. The method comprises: (a) administering to one or more subjects one or more therapeutic interventions, wherein the subject is a model of a physiologic state and the taxa targeted by the immune system in the subject are known; (b) identifying one or more taxa targeted by the immune system of the subject after administration of the therapeutic intervention to the subject, wherein the one or more taxa targeted by the immune system are identified by the method of any of the methods of the invention described herein; and (c) comparing the strength of enrichment for each taxon in the detection agent bound population before and after administration of the therapeutic intervention to the subject; wherein a change in the strength of enrichment after administration as compared to before administration of the therapeutic intervention indicates the therapeutic intervention was effective at modulating the immune response.

Another aspect of the invention encompasses a method for identifying a physiological state of a subject. The method comprises (a) obtaining a biological sample from the subject comprising microorganisms from different taxa; (b) mixing the sample with one or more detection agents; (d) sorting the sample into two populations: a detection agent bound microorganism population and an unbound microorganism population; (e) identifying the taxonomic composition of the detection agent bound microorganism population and the unbound microorganism population; (f) comparing the taxonomic composition of the detection agent bound microorganism population to the unbound microorganism population; (g) calculating a strength of enrichment for each taxon in the detection agent bound population, wherein a strength of enrichment value greater than zero indicates enrichment in the detection agent bound population; (h) comparing the taxa that are enriched in the detection agent bound population of the subject to the taxa enriched in the detection agent bound population of one or more reference subjects; and (i) identifying the physiological state of the subject when the taxa enriched in the detection agent bound population of the subject is statistically similar to the detection agent bound population of a reference subject.

Another aspect of the invention encompasses a method of identifying one or more taxa targeted by the immune system of a subject. The method comprises (a) obtaining a biological sample comprising microorganisms from different taxa from the subject; (b) mixing the sample with one or more detection agents; (c) sorting the sample into two populations: a detection agent bound microorganism population and an unbound microorganism population; (d) identifying the taxonomic composition of the detection agent bound microorganism population and the unbound microorganism population; (e) comparing the taxonomic composition of the detection agent bound microorganism population to the unbound microorganism population; and (f) calculating a strength of enrichment for each taxon in the detection agent bound population; wherein a strength of enrichment value greater than zero indicates enrichment in the detection agent bound population and targeting by the immune system.

Another aspect of the invention encompasses a method of screening for a therapeutic intervention effective at modulating a subject's immune response to one or more taxa. The method comprises: (a) providing a plurality of therapeutic interventions; (b) administering to a number of subjects the therapeutic interventions, wherein (i) the subject is a non-human animal model of a physiologic state and the taxa targeted by the immune system in the subject are known, and (ii) the number of subjects is equal to or greater than the number of therapeutic interventions; (c) identifying one or more taxa targeted by the immune system of the subject after administration of the therapeutic intervention to the subject, wherein the one or more taxa targeted by the immune system are identified by any method disclosed herein; and comparing the strength of enrichment for each taxon in the detection agent bound population before and after administration of the therapeutic intervention to the subject; wherein a change in the enrichment of a taxon after administration as compared to before administration of the therapeutic intervention indicates the therapeutic intervention was effective at modulating the subject's immune response to that taxon.

Another aspect of the invention encompasses a method for determining the effectiveness of a therapeutic intervention at modulating the immune response in a subject. The method comprises: (a) identifying one or more taxa targeted by the immune system of the subject before and after administration of the therapeutic intervention to the subject, wherein the one or more taxa targeted by the immune system are identified by any method disclosed herein; and (b) comparing the strength of enrichment for each taxon in the detection agent bound population before and after administration of the therapeutic intervention to the subject; wherein a change in the strength of enrichment after administration as compared to before administration of the therapeutic intervention indicates the therapeutic intervention was effective at modulating the immune response.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 graphically illustrates the purification of a specific bacteria based on its binding to a host immunoglobulin A (IgA). In particular, FIG. 1 shows microorganism separating FACS (BugFACS) enrichment of B. thetaiotaomicron from a mixture containing E. rectale and B. thetaiotamicron. The fraction of 16S rRNA reads attributable to B. thetaiotaomicron prior to enrichment was approximately 0.1%. After sorting, based on the presence of a monoclonal antibody specific to B. thetaiotaomicron, 80% of the total reads were attributable to this bacterial species (IgA+ fraction) while B. thetaiotaomicron was nearly absent from the fraction with no detectable IgA binding (IgA− fraction).

FIG. 2 graphically illustrates that bacteria capable of causing disease and taxa with protective disease mitigating properties can be isolated in a viable form and identified using methods of the invention. Specifically, FIG. 2 shows the phenotype of mice colonized with (i) a BugFACS sorted IgA-positive population of microbes isolated from the feces of gnotobiotic mice fed a micro- and macronutrient deficient diet representative of that consumed by human populations living in Malawi and containing a transplanted fecal microbiota from a Malawian co-twin with kwashiorkor in a twin-pair discordant for this form of severe acute malnutrition (abbreviated Kwash-Mal IgA+), or (ii) assorted IgA+ population from mice fed a Malawi diet and harboring a transplanted gut microbiota from that individual's healthy co-twin (Healthy-Mal IgA+) or (iii) a mixture of the two IgA+ populations (‘Mix’). IgA-positive microbes were isolated and gavaged into mice as described in the examples. All mice were fed the Malawi diet and weights were obtained daily. (A) Significant weight loss in mice gavaged with the ‘Kwash-Mal IgA+ population. The weight loss phenotype was ameliorated for several days by mixing the Healthy-Mal IgA+ population with the Kwash-Mal IgA+’ population. * p<0.05, **p<0.001 (Student's t test, comparing mice with the Healthy-Mal IgA+ population or the Mix to mice harboring the transplanted Kwash-Mal IgA+ population). (B) Increased mortality in mice receiving a ‘Kwash-Mal’ IgA+ population, rescued with a Healthy-Mal IgA+ population **p<0.01 (Chi-square test, comparing Healthy-Mal IgA+ or Mix to Kwash-Mal IgA+). The number of mice used is indicated: experiments were repeated on two independent occasions with 5-10/treatment group.

FIG. 3 shows “volcano” plots demonstrating a significant enrichment of the family of bacteria, Enterobacteriaceae, in the fecal microbiota of a cohort of Malawian twin pairs that were discordant for kwashiorkor. Enterobacteriaceae were significantly enriched in the co-twins diagnosed with kwashiorkor (FIG. 3A) and in their healthy co-twins (FIG. 3B). The plots show the negative logarithm of p value (determined by paired t test) on the y-axis and the logarithm of the ratio of the representation of a given taxon in the IgA positive population to its representation in the IgA negative population. Data were obtained from bug FACS analysis of human fecal samples.

FIG. 4 depicts graphs showing that Bug FACS reproducibly enriches IgA bound microbes. Four different mixtures of B. thetaiotaomicron and E. rectale were created with varying proportions of each taxon. Mixtures of each taxon were stained first with a monoclonal anti-B. thetaiotaomicron IgA antibody, followed by a polyclonal goat anti-mouse secondary conjugated to DyLyght 649. Finally, a DNA stain (SytoBC) was added to help distinguish bacteria from other particles. The four pie charts in (A), numbered (i), (ii), (iii), and (iv), depict ratios of B. thetaiotaomicron and E. rectale in the input. (B) An “Input” fraction was collected based on FSC (x-axis) and SSC (y-axis) characteristics. (C) Bacteria collected in the Input Fraction were then gated based on binding to SytoBC (x-axis) and PerCP-Cy5.5 (y-axis). SytoBC positive bacteria (within the boxed area in (C)) were then sorted by IgA in (D). (D) IgA bound (IgA+) and unbound bacteria (IgA−) were collected for each fraction. Proportional representation of each taxon (B. thetaiotaomicron and E. rectale) within each IgA fraction are depicted with the pie charts for each mixture of bacteria ((i)-(iv), where the labels correspond to the input in (A)). For each mixture of bacteria, the pie chart and FACS image on the left are IgA negative, and the pie chart and FACS image on the right are IgA positive. (E) Statistically significant deenrichment (E. rectale) or enrichment (B. thetaiotaomicron) can be calculated by comparing the proportion representation of each taxa in the IgA− and IgA+ fractions for each sample and calculating a paired test (Wilcoxon paired test). Data from three separate experiments are shown. (F) An IgA index score can be calculated for each taxon based on the proportional representation of that taxon within the IgA+ and IgA− fraction. This IgA index ranges from −1 to +1, with a negative score indicating that the taxa is found at a higher abundance in the IgA− fraction and positive score indicating that it is found at a higher abundance in the IgA+ fraction. In order to calculate an IgA index score for taxa that has an observed relative abundance of zero, a pseudocount is added to both relative abundance terms. The bubble plots in (E) and (F) are a summary of the statistical significance of IgA (de-)enrichment and an average of the calculated IgA index for a single taxon across a group of samples.

FIG. 5 depicts graphs and illustrations showing the experimental design for Example 6. (A) Pulverized fecal specimens from twin pairs discordant for kwashiorkor were used to generate humanized “Kwashiorkor” and “Healthy” mice. These mice were fed either a standard low fat, plant polysaccharide-rich mouse chow diet or a macro- and micronutrient deficient “Malawian” diet. (B) IgA bound bacteria were recovered from the fecal microbiota of humanized gnotobiotic mice (shown in (A)) using fluorescence assisted flow cytometry (FACS), gating on a fluorescent DNA-specific dye (SytoBC, Molecular Probes) and a secondary antibody to mouse IgA conjugated to DyLight 649 (Abcam). Three separate populations of bacteria were isolated from each fecal specimen: the “Input” population is collected from a gate with particles with the size and granularity (forward and side scatter properties) of bacteria (indicated in the boxed area of (B). (C) The “IgA−” and “IgA+” populations both bound SytoBC (indicated in the boxed area of (C) but were differentiated by the presence (IgA+) or absence (IgA−) of IgA (D) and (E). Rag1−/− mice, which lack the ability to make antibodies, including IgA, had no discernable IgA+ microbial population (F). (D,E) Gnotobiotic mice colonized with the IgA+ fraction purified from the fecal microbiota of KM or HM mice are labeled KMIgA+ (D) and HMIgA+ (E), respectively. Mice colonized with an equal mixture of bacteria from the IgA+ fractions of KM and HM mice are labeled MixIgA+(E). (G) Bacteria isolated from IgA+, IgA−, and Input fractions using BugFACS were also subjected to V2-16S rRNA amplicon sequencing to identify taxa that are targets of a host IgA response.

FIG. 6 depicts graphs showing. (A) Mice humanized with a kwashiorkor microbiota (K) and fed a macro- and micro-nutrient deplete Malawian diet (M) show significant weight loss compared to mice that are fed a macro- and micronutrient replete standard diet (S) or mice colonized with a healthy co-twin's microbiota (H) fed the same diet. For clarity: KM=kwashiorkor donor microbiota and Malawian diet; KS=kwashiorkor donor microbiota and standard diet; HM=healthy donor microbiota and Malawian diet; HS=healthy donor microbiota and standard diet. (B) 16S V2 rRNA amplicon pyrosequencing of the fecal microbiota of humanized mice shows that the largest contributor to variance, as determined by Principal Coordinate Analysis (PCoA) of unweighted UniFrac distances between the fecal communities of humanized mice, is ‘donor microbiota’. (C) Diet is the second largest contributor to variance between mice. Data were compiled from two separate experiments using the same twin pair 57. Fecal 16S V2 rRNA data shown in (B) and (C) are from 10 and 21 days after colonization, averaged across each mouse.

FIG. 7 graphically depicts results from V2-16S rRNA amplicon pyrosequencing of BugFACS fractions, which identifies diet- and microbiota-associated differences in the targets of IgA responses in humanized gnotobiotic mice. Mice were humanized as described in FIG. 5A and samples taken for IgA analysis 12-15 days after colonization. Results shown are combined from two independent experiments. (A) IgA Index for Enterobacteriaceae is graphed on the y-axis. KM mice had a statistically greater enrichment of the taxon Enterobacteriaceae in the IgA+ fraction compared to mice receiving the standard mouse chow diet (KS) or mice receiving a microbiota from a healthy co-twin on either diet (HM and HS). (B) Mice receiving a microbiota from a healthy co-twin on either diet (HM and HS) had greater IgA enrichment of Verrucomicrobiaceae (as per the IgA Index on the y-axis) than mice receiving the microbiota of a co-twin with kwashiorkor on either diet (KM and KS) (Wilcoxon Rank Sum test; **p<0.01; ***p<0.001; ****, p<0.0001) (C) Analysis of IgA responses to family level taxa in humanized mice. Each column represents a different group of humanized mice and each row depicts the family-level taxonomic analysis of enrichment in the IgA+ fraction. The color of the circles represent the average direction of enrichment: red and yellow indicate that the taxon is enriched in the IgA+ fraction, while blue or green denotes enrichment of that taxon in the IgA− fraction. The diameter of a given circle represents the average magnitude of enrichment (see FIG. 4). Red and blue indicate statistically significant enrichment, with darker colors indicating greater significance (significance is assumed for p<0.05 as determined by paired Wilcoxon test), while green and yellow indicate that enrichment of the taxon was not statistically significant.

FIG. 8 graphically depicts results of V2 16S rRNA Sequencing of BugFACS fractions providing information about mouse IgA specificity. (A) Average relative abundance of Enterobacteriaceae and (B) Verrucomicrobiaceae indicate that these taxa are present in all humanized mouse experimental groups. (C) Weighted UniFrac comparison of BugFACS fractions from a single sample demonstrate predicted relationships between the fractions. The IgA+ and IgA− fractions are least similar to one another while IgA− and input fractions are most similar. The similarity between IgA+ and Input fractions is intermediate. (D) Unweighted UniFrac distances show a similar pattern as in (C), but are less pronounced. Input (E) IgA− (F) and IgA+(G) fractions obtained using BugFACS maintain the closest similarity, as measured by weighted UniFrac to (in descending order): (1) the mouse from which the fractions were derived (red); (2) mice sharing the same microbiota and diet (yellow); (3) mice sharing the same microbiota; (4) mice sharing the same diet (blue); and (5) all mice in the experiment.

FIG. 9 depicts graphs showing transplantation of the IgA+ fraction purified from kwashiorkor microbiota results in increased weight loss and mortality in recipient gnotobiotic mice. All mice were fed a Malawian diet starting one week prior to colonization and gavaged with the IgA+ fraction of bacteria purified from the fecal microbiota of humanized mice sampled 42 d after colonization. Results represent combined data from two independent experiments. (A) KM^{IgA+} mice (n=20) experienced significantly more mortality compared to HM^{IgA+} mice (n=15) and Mix^{IgA+} mice (n=10 mice) over the 13 day course of the experiment (Chi-squared test). (B) Surviving KM^{IgA+} mice experienced more weight loss than HM^{IgA+} mice. Mix^{IgA+} mice had an intermediate phenotype (t-test; * comparison to KM^{IgA+} mice, + comparison to HM^{IgA+} mice). (C) Clostridium scindens was found in HM, HM^{IgA+} and Mix^{IgA+} mice, but was not detected in KM or KM^{IgA+} animals (Chi-Square test). (D) Three groups of mice were gavaged with the IgA+ fraction recovered by BugFACS from the fecal microbiota of surviving KM^{IgA+} mice. The first group received no intervention (KM^{F2IgA+} mice, n=10). The second group received a mixture of live Clostridium scindens and Akkermansia muciniphila 24 h before introduction of the KMI^{gA+} microbiota (CsAm+KM^{F2IgApos}, n=15). In the third group, heat-killed C. scindens and A. muciniphila were gavaged 24 h prior to introduction of KM^{IgA+} microbiota (HK CsAm+KM^{F2IgA+}). (E) CsAm+KM^{F2IgA+} mice had reduced mortality when compared to either KM^{F2IgA+} or HK CsAm+KM^{F2IgA+} mice. (*, +p<0.05; **p<0.01; ***p<0.005; ****, #p<0.0005).

FIG. 10 depicts graphs showing the results of transfer of KM^{IgA+} and HM^{IgA+} microbiota into germ-free mice. (A) Comparison of the composite weighted UniFrac distance between IgA+ and IgA− BugFACS fractions from humanized KM and HM mice, and fecal microbiota of KM^{IgA+} (green) or KM^{IgA+} (red) mice (sampled 13 days after gavage) reflects both the microbiota of origin and the BugFACS fraction from which it originated. (B) Rarefaction curves of 97% ID OTUs identified from fecal V2 16S rRNA sequencing of samples obtained from humanized KM and HM mice (42d after colonization) and mice receiving a KM^{IgA+}, HM^{IgA+} or Mix^{IgA+} fraction (13d after colonization). Mice receiving a IgA+ consortium demonstrate lower alpha diversity compared to mice receiving the complete human microbiota (C,D) PCoA of 16S rRNA data. KM^{IgA+} (green) mouse microbiota are distinct from HMIgA+ (red) microbiota. MixIgA+ (orange) microbiota appear most similar to HM^{IgA+} microbiota while KM^{IgA+}+CsAm have an intermediate relationship to both KM^{IgAPos} and HM^{IgApos} microbiota. The effect of microbiota source can be seen in both PC1 (C) and PC2 (D) which together account for 44.7% of the 16S rRNA weighted UniFrac variance. (E) PCoA of unweighted UniFrac distances demonstrate that the fecal microbiota of mice colonized with KM^{IgA+} and HM^{IgA+} consortia are distinct from one another. The fecal microbiota of Mix^{IgA+} mice appears intermediate between KM^{IgA+} and HM^{IgA+} while KM^{IgA+}+CsAm appears most similar to KM^{IgA+} animals from which it originated. (F) KM^{IgA+} mice fed a Malawian diet (green) lose more weight than mice colonized with the same microbiota fed a standard mouse chow diet (brown). Mice receiving IgA+ microbes from a mouse humanized with the same microbiota but fed a standard mouse chow also lose less weight than KM^{IgA+} mice regardless if they were fed the same diet (cyan) or a standard mouse chow (purple).

FIG. 11 depicts graphs showing the results of BugFACS extended to human fecal specimens. (A) Staining of human fecal specimens with a goat polyclonal anti-mouse IgA demonstrates very little non-specific staining. (B) Staining of fecal specimens with a goat polyclonal anti-human IgA demonstrates robust staining. (C) BugFACS of the same fecal sample, conducted on separate days, demonstrates reproducible identification of microbes bound (or unbound) to IgA. Nine human fecal samples were prepared, stained, sorted, subjected to BugFACS and the purified fractions sequenced as described on separate days. The IgA index calculated for each family-level taxon within the first sample (replicate 1) was compared to the IgA index calculated for the same taxon in the replicate 2 samples. Therefore, each point represents a comparison of a single taxon between replicate 1 and 2. (D) The amount of fecal material used to perform BugFACS (in grams) is plotted against the percentage of IgA+ events. There was no statistically significant correlation. (E,F) V2-16S rRNA sequencing of human-derived BugFACS fractions. Patterns of relatedness as determined by weighted (E) and unweighted (F) UniFrac between fractions were identical to that observed in mouse specimens (see FIG. 8).

FIG. 12. depicts graphs showing Enterobacteriaceae are targeted by the IgA response in children with kwashiorkor. (A) IgA responses against select family-level taxa shown for co-twins with kwashiorkor (left) or co-twins who remained healthy (right). Data from five time points are shown: the first column for each co-twin represents samples taken 1-3 months before the diagnosis of kwashiorkor (“Pre-diagnosis”). The second column represents samples taken at the time of diagnosis (“Diagnosis”). The third and fourth columns are samples taken after 2 or 4 weeks of treatment with RUTF, respectively. The fifth column was taken 1 month after the completion of RUTF therapy. The final column is a combined IgA enrichment for each taxon across all time points. (B) At the time of diagnosis, the calculated IgA index score against Enterobacteriaceae was higher in twins with kwashiorkor than in twin pairs who were concordant for healthy status. The IgA index score against Enterobacteriaceae was averaged for each co-twin sample from concordant healthy pairs obtained between 6 and 24 months of age to allow comparison to discordant twins of varying ages at the time of diagnosis of kwashiorkor. Pink or green points represent discordant co-twin samples selected for microbial adaptive transfer (see (D)) (C) Treatment with RUTF results in a decrease in the IgA index score against Enterobacteriaceae. Data obtained while on RUTF represents the average IgA index score of samples procured after 2 and 4 weeks of RUTF treatment. (D) Mice colonized with a purified IgA+ consortium of microbes originating from an individual with kwashiorkor lost more weight than mice colonized with the corresponding IgA+ fraction from the healthy co-twin, or with a mixture of the two fractions. IgA+ fractions obtained directly from twin pair 46 were used to colonize mice with a purified kwashiorkor fecal IgA+ consortium, a healthy IgA+ consortium or an equal mixture of the two preparations (Mix IgA+) (n=6 mice/group). All recipient gnotobiotic mice were fed the Malawian diet starting 1 week before gavage and were weighed daily until sacrifice 13d post-gavage. (t-test; *, #p<0.05; **, #p<0.01; ***p<0.001; ****p<0.0001; “*” used for comparison between animals colonized with a kwashiorkor co-twin IgA+ and healthy co-twin IgA+ fraction, “#”, comparison between kwashiorkor IgA+ and mix IgA+ colonized animals.) (E) BugFACS analysis of fecal samples revealed that responses against Bifidobacteriaceae increased as a function of the age regardless of the health status of the child, suggesting at least some ordered ontogeny of IgA responses. Consistent with this idea, the absolute fraction of IgA+ events decreased with age.

### DETAILED DESCRIPTION OF THE INVENTION

In accordance with the present invention, a method for identifying and isolating microorganisms that are targets of immune responses in a subject has been discovered. Identifying and retrieving microorganisms, in a viable form, that are targets of the subject's immune response has diagnostic and therapeutic value. These microorganisms could provide health benefits, either when administered as live organisms (probiotics), or in combination with nutrient supplements (as synbiotics), or for identifying compounds that promote their growth (prebiotics) or that inhibit or prevent their growth (antibiotics).

### I. Methods of Isolating and Identifying Microorganisms Targeted by the Immune System in a Subject.

In one aspect, methods of the invention include isolating microorganisms targeted by the immune system of a subject. The microorganisms may be viable or unviable. In preferred embodiments, the microorganisms are viable. Maintaining the viability of the cells is desired so that (i) their biological properties and products can be characterized using in vitro and in vivo assays; and (ii) they and their products can be propagated and subsequently used as therapeutic and/or diagnostic agents. As used herein, the term “microorganism” refers to bacteria, fungi, yeasts, archaea, protists, and viruses. Such methods include the steps of obtaining a biological sample, mixing the sample with detection agents, and sorting the microorganism populations according to the bound state of the detection agent. The methods may also include comparing the compositions of the sorted microorganism populations, calculating the strength of enrichment of the bound population, identifying the microorganisms contained in the populations, correlating the identified microorganisms to a physiological state, or other methods known in the art.

In another aspect, methods of the invention include identifying one or more groups of microorganisms targeted by the immune system of a subject. Preferably, the microorganisms are viable. Microorganisms may be identified, or grouped, on one or more taxonomic levels (e.g. species, genus, family, order, class, and/or phylum) as described below. Thus, in another aspect, methods of the invention include identifying or more taxa targeted by the immune system of a subject. Typically, the method comprises: (a) obtaining a biological sample comprising microorganisms from different taxa from the subject; (b) mixing the sample with one or more detection agents; (c) sorting the sample into two populations: a detection agent bound microorganism population and an unbound microorganism population; (d) identifying the taxonomic composition of the detection agent bound microorganism population and the unbound microorganism population; (e) comparing the taxonomic composition of the detection agent bound microorganism population to the unbound microorganism population; and (f) calculating a strength of enrichment for each taxon in the detection agent bound population. A strength of enrichment value greater than zero indicates enrichment in the detection agent bound population and targeting by the immune system.

Typically, the subject is a human or a non-human animal. Non-liming examples of non-human animals include a livestock animal, a companion animal, a lab animal, or a zoological animal. In one embodiment, the subject may be a human. In another embodiment, the subject may be a livestock animal. Non-limiting examples of suitable livestock animals may include pigs, cows, horses, bison, goats, sheep, llamas and alpacas. In yet another embodiment, the subject may be a companion animal. Non-limiting examples of companion animals may include pets such as dogs, cats, rabbits, and birds. In a different embodiment, the animal is a laboratory animal. Non-limiting examples of a laboratory animal may include rodents, canines, felines, and non-human primates. In an alternative embodiment, the subject may be a zoological animal. As used herein, a “zoological animal” refers to an animal that may be found in a zoo. Such animals may include non-human primates, large cats, wolves, and bears.

In another aspect, isolating and/or identifying microorganisms targeted by the immune system of a subject comprises identifying an interaction of the subject's immune system with a microorganism (i.e. an immune system: microorganism interaction or an immune system: microorganism complex). Any such interaction capable of being detected is contemplated herein. Numerous such interactions are well known in the art and are contemplated herein. Non-limiting examples of direct or indirect ways microorganisms interact with the immune system include interactions with immunoglobulins, complement and T-cells. In some embodiments, the immune system:microorganism complex is an immunoglobulin:microorganism complex.

In some embodiments, an immune system: microorganism interaction is detected by the detection of an immunoglobulin. In these embodiments, the immune system:microorganism complex comprises an immunoglobulin. As used herein, the term immunoglobulin (Ig) refers to the glycoproteins of the five main classes (IgA, IgM, IgD, IgE, and IgG), as well as all subclass, types and subtypes for each class. Non-limiting examples of IgG subclasses include IgG1, IgG2, IgG3, IgG4. Non-limiting examples of IgA subclasses include IgA1 and IgA2. Immunoglobulins can also be classified by the type of light chain that they have. Non-limiting examples of light chains may include kappa light chains and lamda light chains. The light chains can also be divided into subtypes based on differences in the amino acid sequences in the constant region of the light chain. Non-limiting examples of lambda subtypes may include lambda 1, lambda 2, lambda 3, and lambda 4. Methods of detecting and distinguishing immunoglobulin classes, subclasses, types and subtypes are known in the art.

One skilled in the art will recognize that the identified microorganisms may be correlated to a specific physiological state based on the immunoglobulin used for detection. For example, if IgG or IgM is used, the microorganism may affect or be effected by inflammation. If IgE is used, the microorganism may be involved in development of allergy. If IgA is used, the microorganism may be found in mucosal areas, such as the gut, respiratory tract and urogenital tract, and affect these mucosal barriers. In exemplary embodiments, the immunoglobulin detected is IgA and identified microorganisms correlate to mucosal barrier function. In other exemplary embodiments, the immunoglobulin detected is IgA and identified microorganisms correlate to gastrointestinal mucosal barrier function.

### A. Biological Samples

Biological samples appropriate for use with the invention include any biological sample isolated from a subject comprising at least one immune system: microorganism complex. Generally speaking, a biological sample will comprise more than one immune system: microorganism complex. The sample may also comprise microorganisms from more than one species, genus, family, order, class, and/or phylum. In some embodiments, the biological sample comprising at least one immune system: microorganism complex further comprises an immunoglobulin: microorganism complex. The immunoglobulin:microorganism complex may comprise any of the classes of immunologlobulin including, but not limited to, IgA, IgM, IgG, IgD, and IgD.

In some embodiments, the biological sample is obtained from a mucosal lining of a subject. Non-limiting biological samples may include those from gastrointestinal, vaginal, genitourinary, pulmonary, skin, oral, nasopharyngeal, eye, and sinus areas. Suitable biological samples include those in a dry or liquid state. Contemplated within the phrase “biological samples obtained from a mucosal lining” include both samples of the mucosal lining itself as well as samples that were in contact with the mucosal lining. Non-limiting examples of samples that were in contact with the mucosal lining include fecal matter, biological fluids (for example, luminal contents recovered from the gastrointestinal tract, saliva, urine, vaginal secretions, tears, sweat, mucus, sputum), as well as fluids recovered during medical procedures (for example, lavages). In a preferred embodiment, the biological sample is a fecal sample. In another preferred embodiment, the biological sample is a biological fluid. In another preferred embodiment, the biological sample is a fluid recovered after lavaging a subject.

As will be appreciated by a skilled artisan, the method of collecting a biological sample can and will vary depending upon the nature of the biological sample. Any of a variety of methods generally known in the art may be utilized to collect a biological sample. Generally speaking, the method preferably maintains the integrity of the sample such that immune system: microorganism interaction may be accurately detected according to the invention. Additionally, the method preferably maintains the viability of the microorganism in the immune system:microorganism complex.

Methods of obtaining samples of the mucosal lining are known in the art, and may include, but are not limited to, tissue biopsy or dissection of the tissue after removal from a subject. Methods of obtaining biological fluids and fluids recovered during medical procedures are known in the art. In a preferred embodiment, a biological sample is obtained from a gastrointestinal area. In an exemplary embodiment, a biological sample comprises fecal matter.

A biological sample may be further processed in order to facilitate its use in downstream steps of the method, provided the immune system: microorganism interaction is not disrupted. Such methods are well known in the art and further detailed in the Examples.

### B. Detection Agents

Detection agents suitable for use with the invention include any detection agents capable of identifying an interaction of the subject's immune system with a microorganism. Immune system: microorganism interactions contemplated are described above. Typically, the detection agent recognizes and is capable of binding to the immune system component of the immune system: microorganism complex in a biological sample. For example, the detection agent may be able to specifically bind to an immune system: microorganism complex comprising an immunoglobulin. The biological sample may or may not contain other microorganisms that are not bound by or interacting with the immune system. For example, the biological sample may contain other microorganisms that are not bound by immunoglobulins. A detection agent specific for the immune system component provides the ability to sort the sample into two populations based on the presence or absence of the immune system component in downstream steps. Non-limiting examples of suitable detection agents include antibodies, aptamers, molecular probes, proteins, peptides, DNA, RNA, small molecules, and combinations thereof. Further, any detection agent known in the art or yet to be discovered may also be suitable.

In some embodiments the detection agent is an antibody. As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety). In an exemplary embodiment, the antibody is an anti-IgA antibody. In another exemplary embodiment, the antibody is an anti-IgG antibody. In still another exemplary embodiment, the antibody is an anti-IgM antibody. In yet another exemplary embodiment, the antibody is an anti-IgE antibody. In an additional exemplary embodiment, the antibody is an anti-IgD antibody.

In some embodiments the detection agent is an aptamer. As used herein, the term “aptamer” refers to a polynucleotide, generally an RNA or DNA that has a useful biological activity in terms of biochemical activity, molecular recognition or binding attributes. Usually, an aptamer has a molecular activity such as binding to a target molecule at a specific epitope (region). It is generally accepted that an aptamer, which is specific in its binding to a polypeptide, may be synthesized and/or identified by in vitro evolution methods. Means for preparing and characterizing aptamers, including by in vitro evolution methods, are well know in the art (See, e.g. U.S. Pat. No. 7,939,313; herein incorporated by reference in its entirety). In an exemplary embodiment, the aptamer specifically binds to IgA. In another exemplary embodiment, the aptamer specifically binds to IgG. In still another exemplary embodiment, the aptamer specifically binds to IgM. In yet another exemplary embodiment, the aptamer specifically binds to IgE. In an additional exemplary embodiment, the aptamer specifically binds to IgD.

A skilled artisan will appreciate that the immunoglobulins vary between species, and that the methods of the invention contemplate the use of species-specific detection agents where appropriate. For example, if the subject is a mouse, an anti-mouse IgA antibody may be used. Similarly, if the subject is a human, an anti-human IgA antibody may be used. Other species-specific antibodies known in the art are also contemplated.

Detection agents may be labeled for detection. The term “label”, as used herein, refers to any substance attached to detection agent, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, streptavidin, protein A, protein G, antibodies or fragments thereof, polyhistidine, Ni²⁺, FLAG tag, myc tags, heavy metals, and enzymes (including alkaline phosphatase, peroxidase, and luciferase). In some embodiments, the detection agent is labeled with a fluorophore.

In general, a biological sample is contacted with a detection agent under conditions effective to allow for formation of a complex between the detection agent and the immune system: microorganism complex. This interaction typically occurs in solution with mixing (i.e. agitation). Detection agents may also be attached to a solid support. Non-limiting examples of suitable surfaces include microtitre plates, test tubes, beads, resins and other polymers. Methods of labeling and detection based on a label, both in solution and using a solid support, are well known in the art. Further detail may also be found in the Examples.

In some embodiments, it may also be desirable to label the microorganisms in the sample. Methods for labeling microorganisms are known in the art. For example, an antibody or an aptamer specific for the microorganism may be used. Alternatively, a nucleic acid dye or stain may be used. Suitable nucleic acid dyes and stains are known in the art and are commercially available. In some embodiments, the nucleic acid dye may be SytoBC. Such a dye may bind to molecules present in the microbe (e.g. DNA) without rending the organism unviable.

C. Sorting the Sample into Two Populations: a Detection Agent Bound Microorganism Population and an Unbound Microorganism Population

According to the methods of the invention, a sample may be sorted into two populations: a detection agent bound microorganism population and an unbound microorganism population. The detection agent bound and unbound microorganisms may be sorted using any method known in the art. Suitable sorting methods include those that efficiently sort bound and unbound microorganisms into two or more populations based on the presence or absence of the detection agent. In this way, the microorganisms comprising the biological sample are sorted based on the presence or absence of an interaction with the subject's immune system (i.e. the presence or absence of an immune system: microorganism complex). The methods may or may not result in viable microorganism. In some embodiments, the methods are capable of sorting microorganisms such that the microorganisms remain viable. In other embodiments, the methods are capable of sorting microorganisms such that the microorganisms do not remain viable.

Preferably, the efficiency of sorting is such that more of the microorganisms in the detection agent bound group are bound to a detection agent than not bound, and more of the microorganisms in the unbound group are not bound to a detection agent than bound to a detection agent. Non-limiting examples of sorting methods include fluorescence activated cell sorting (FACS or BugFACS), immunoprecipitation, antibody-bead conjugated separation, and combinations thereof.

### D. Identifying the Taxonomic Composition of Each Population

According to the methods of the invention, the taxonomic composition of the detection agent bound microorganism population and the unbound microorganism population may be identified and recovered in a viable form from a complex mixture of organisms that together comprise a microbial community present in a given body habitat of a subject. Identification may be done at the species, genus, family, order, class, or phylum level, or any combination thereof. In some embodiments, identification is done at the species level. In other embodiments, identification is done at the genus level. In still other embodiments, identification is done at the family level. In yet other embodiments, identification is done at the order level. In additional embodiments, identification is done at the class level. The sorted microorganisms can be identified using any method known in the art or yet to be discovered. Non-limiting examples of suitable identification methods include culture-dependent and culture-independent methods.)

In some embodiments, the taxonomic composition may be identified by culture-dependent methods. The phrase “culture-dependent methods” refers to growing isolated microorganisms with different culture media and environments. Culture-dependent methods are well known in the art and contemplated herein. For example, standard anaerobic techniques to minimize oxygen exposure should be used to recover and culturing gut microorganisms. Exemplary culture-dependent methods include, without limitation, gelysate agar to detect aerobic mesophillic flora, MRS agar to detect lactic acid bacteria and bifidobactria, mannitol sugar agar, kanamycin-esculin to detect enterococci, Baird-Parker with egg yolk tellurite emulsion to detect Staphylococcus aureus, and malt extract to detect yeast and mold.

In other embodiments, the taxonomic composition may be identified by culture-independent methods. Exemplary culture-independent methods include, without limitation, denaturing gradient gel electrophoresis (DGGE); DNA sequence identification methods such as sequencing the phylogenetic marker gene, 16S rDNA, or performing shotgun sequence of DNA isolated from the sorted population; metagenomic methods such as sequencing amplified rRNA sequences; and combinations thereof. Such methods are known in the art, and further detailed in the examples. In a preferred embodiment, the microorganisms are identified by sequencing amplified rRNA sequences and comparing the sequence to known sequences.

### E. Calculating a Strength of Enrichment

According to the methods of the invention, a strength of enrichment is calculated for each taxon in the detection agent bound population. Generally speaking, strength of enrichment calculations may be used to make a comparison both within a population and across two populations. Greater detail of suitable strength of enrichment calculations are described below and in the Examples.

For example, a strength of enrichment calculation may be used to determine the efficiency of the sorting method or it may be used to identify those taxa whose representation are greater (enriched) in the detection agent positive (bound) population compared to the detection agent negative (unbound) population. In the former, pre-sort and post-sort control samples can be analyzed to track contamination. The data from the control samples can be used to correct test sample data. One way this could be done is to remove from the test sample data microorganisms or taxa identified in the control samples. In the latter example, when coupled to repeated measures (either of the same sample or over a population), a p value can be generated that indicates the degree of confidence for that taxa being significantly enriched in the detection agent positive population. Additionally, appropriate controls can be used for non-specific binding of the detection agent, which may be a source of false positive taxa. For example, with an antibody as a detection agent, a control sample from Rag1−/− mice that lack B-cells and are unable to produce antibody to assay for specificity of binding can be analyzed.

In some embodiments, the strength of enrichment can be calculated by analyzing the linear relationship (for a given taxon) between detection agent positive, detection agent negative and input populations. The slope of this line, with intercept equal to zero, can be calculated by:

$$\frac{log ( {IgApositive}_{taxon} / {IgAnegative}_{taxon} )}{- log ( {Input}_{taxon} )}$$

This number represents the strength of enrichment of a taxon in the detection agent positive fraction with any value greater than 0 representing enrichment in the detection agent positive population. The strength of enrichment, in turn, is determined by multiple factors including the amount of detection agent present, the strength of detection agent binding, and factors related to the efficiency of sorting, but is not dependent on the abundance of the taxa within the sample, allowing cross-sample comparisons.

In other embodiments, statistically significant deenrichment or enrichment can be calculated by comparing the proportion representation of each taxa in the detection agent negative (−) and detection agent positive (+) fractions for each sample and calculating a paired test.

In other embodiments, strength of enrichment calculations can be presented as a detection agent index score that can be calculated for each taxon based on the proportional representation of that taxon within the detection agent negative (−) and detection agent positive (+) fraction. This detection agent index ranges from −1 to +1, with a negative score indicating that the taxon is found at a higher abundance in the detection agent negative (−) fraction and positive score indicating that it is found at a higher abundance in the detection agent positive (+) fraction. In order to calculate a detection agent index score for taxa that have an observed relative abundance of zero, a pseudocount is added to both relative abundance terms. Bubble plots, or other graphical representations, can be used to present a summary of the statistical significance of detection agent negative (de-)enrichment and an average of the calculated detection agent index for a single taxon across a group of samples.

In a preferred embodiment, the detection agent is specific for IgA antibody and the strength of enrichment calculation is an IgA index. The IgA index may be calculated by:

$$IgA index = - \frac{log ( {IgA}_{taxon}^{+} ) - log ( {IgA}_{taxon}^{-} )}{log ( {IgA}_{taxon}^{+} ) + log ( {IgA}_{taxon}^{-} )}$$

where IgA⁺ₜₐₓₒₙ and IgA⁻ₜₐₓₒₙ are the relative abundances of taxon in the BugFACS purified IgA positive and IgA negative fractions, respectively. An IgA index greater than zero indicates enrichment in the IgA⁺ population and targeting by the immune system.

In another preferred embodiment, the detection agent is specific for IgG antibody and the strength of enrichment calculation is an IgG index. The IgG index may be calculated similar to the IgA index above.

In another preferred embodiment, the detection agent is specific for IgM antibody and the strength of enrichment calculation is an IgM index. The IgM index may be calculated similar to the IgA index above.

In another preferred embodiment, the detection agent is specific for IgD antibody and the strength of enrichment calculation is an IgD index. The IgD index may be calculated similar to the IgA index above.

In another preferred embodiment, the detection agent is specific for IgE antibody and the strength of enrichment calculation is an IgE index. The IgE index may be calculated similar to the IgA index above.

### F. Methods of Use

Methods for isolating and identifying microorganisms or groups of microorganisms targeted by a subject's immune system have a variety of uses. Such uses include identifying host-bacterial relationships, diagnosing physiologic or pathogenic states based on the microorganisms isolated, identifying pathogens, identifying pathogens from a complex mixture of microorganisms, identifying microorganisms that have preventative or therapeutic effects, identify microorganisms that are capable of modulating the immune system, defining mucosal barrier function as a function of age, physiologic states, metabolic phenotypes, other host parameters including environmental exposures of various types (e.g. food), and other uses.

In one aspect, the methods of the invention may be used to identify properties of microorganisms that may promote health or treat disease. In one aspect, the methods of the invention may be used to identify properties of microorganisms that respond to different dietary components in ways that promote nutritional health. In another aspect, the methods of the invention may be used to identify properties of microorganisms that may be used in diagnostic or therapeutic applications.

In another aspect, methods of the invention may further comprise culturing the detection bound microorganism population. Suitable methods of culturing isolated microorganism or groups of microorganisms are known in the art. In some embodiments, the method of culturing is selected from the group consisting of (i) inoculating the detection agent bound microorganism population into a germ-free (i.e. gnotobiotic) animal, (ii) growing the detection agent bound microorganism population in vitro using standard techniques, and (iii) a combination thereof.

Germ-free animals (gnotobiotic animals) inoculated with a detection agent bound microorganism population may be provided a specific diet such that the microorganisms can be propagated in vivo. The specific diet may resemble that of the host donor or systematically manipulated versions of the host donor diet. Also, the transplanted microorganisms may be subsequently retrieved from the gnotobiotic animals, either by periodic collection of feces, or at the time of sacrifice by sampling along the length of the intestine. The retrieved microorganisms may be cultured such that their growth requirements and metabolic properties can be defined, the genomes characterized, and for various other purposes known in the art or described herein. Suitable gnotobiotic animals include any known in the art. Exemplary gnotobiotic animals include, without limitation, pigs and mice.

### II. Methods of Detecting a Physiological State in a Subject

The Applicants have shown that the identification of microorganisms targeted by the immune system can be used to detect, identify, characterize or classify a physiological state. This is demonstrated in the Examples for three physiological states: a physiological state of malnutrition, a physiological state of general nutrition, and physiological state associated with a specific-diet. Advantageously, these methods do not rely on the presence or identification of outwards signs or symptoms, which may be subjective or which may not manifest until after a physiological state has developed. Thus, the methods described below expressly contemplate identifying a physiological state before a subject may be aware of the physiological state (i.e. the subject is at risk for a physiological state).

In an aspect, methods of the invention include identifying taxa associated with a physiological state. The one or more microorganisms comprising the taxa may be viable or non-viable. Preferably the one or more microorganisms comprising the taxa are viable. Typically, the method comprises: (a) obtaining a biological sample comprising microorganisms from different taxa from one or more subjects with a physiological state and obtaining the same type of biological sample from one or more controls; (b) mixing each sample with one or more detection agents; (c) sorting each sample into two populations: a detection agent bound microorganism population and an unbound microorganism population; (d) identifying the taxonomic composition of the detection agent bound microorganism population and the unbound microorganism population for each sample; (e) comparing the taxonomic composition of the detection agent bound microorganism population to the unbound microorganism population for each sample; (f) calculating a strength of enrichment for each taxon in the detection agent bound population for each sample, wherein a strength of enrichment value greater than zero indicates enrichment in the detection agent bound population, (g) comparing the taxa that are enriched in the detection agent bound population of the one or more subjects with a physiological state to the taxa enriched in the detection agent bound population of the one or more controls; and (h) identifying enriched taxa that are associated with the physiological state of the one or more subjects and not the control. Steps (a)-(f) of the method are described above in Section II. Steps (g) and (h) are described in further detail below.

In another aspect, methods of the invention include detecting or identifying a physiological state of a subject by identifying the taxa targeted by the subject's immune system. Typically the method comprises: (a) obtaining from the subject a biological sample comprising microorganisms from different taxa; (b) mixing the sample with one or more detection agents; (c) sorting the sample into two populations: a detection agent bound microorganism population and an unbound microorganism population; (d) identifying the taxonomic composition of the detection agent bound microorganism population and the unbound microorganism population; (e) comparing the taxonomic composition of the detection agent bound microorganism population to the unbound microorganism population; (f) calculating a strength of enrichment for each taxon in the detection agent bound population, wherein a strength of enrichment value greater than zero indicates enrichment in the detection agent bound population, (g) comparing the taxa that are enriched in the detection agent bound population of the subject to the taxa enriched in the detection agent bound population of one or more references; and (h) identifying the physiological state of the subject when the taxa enriched in the detection agent bound population of the subject is statistically similar to the detection agent bound population of a reference. Steps (a)-(f) are described above in Section II. Steps (g) and (h) are described in further detail below and in the Examples.

### A. Physiological State

As used herein, the term “physiological state” refers to the physical condition or state of the body. The physical condition or state of the body may be good, and the subject may be described as in good health or free from disease. There may be various physiological states associated with good health. For example, a subject may be otherwise in good health and have increased adiposity. Increased adiposity may be viewed as a desirable outcome for livestock and certain laboratory animals, as may other physiological states known in the art. Alternatively, the physical condition or state of the body may be poor, the body may be diseased, or there may be a disturbance or imbalance of normal functioning of the body, and the subject may be described as having a pathological state. Non-limiting examples of pathological states may include malnutrition, obesity, diseases of the gastrointestinal tract (for example, acute or chronic diarrheal disease including inflammatory bowel diseases (e.g. Crohn's disease and ulcerative colitis) Celiac disease), motility disorders such as irritable bowel syndrome, neoplasia, other diseases or states associated with immune dysfunction, plus disease affecting other mucosal surfaces and their associated immune cell populations (e.g. in the mouth, airways, vagina, and urinary tract). Also included in the definition of physiological state is the physical state of the body as shaped or influenced by the diet or therapeutic interventions. The term “therapeutic intervention” refers to pharmaceutical compositions or drug products comprising an API, a biologic, or a combination thereof, as well as dietary interventions. Non-limiting examples of dietary interventions may be prebiotics, probiotics, synbiotics, caloric restriction, caloric supplementation, food group restrictions (e.g. lactose-free, gluten-free, soy-free, peanut-free, nut-free, wheat-free), or changes in the diet that increase or decrease the amount one or more food group relative to the total amount of food. In some embodiments, the physiological condition is malnutrition. In other embodiments, the physiological condition is good health. In other embodiments, the physiological condition is obesity. In other embodiments, the physiological condition is increased adiposity. In other embodiments, the physiological condition may be Crohn's disease. In other embodiments, the physiological condition may be IBS. In other embodiments, the physiological condition may be IBD. In other embodiments, the physiological condition may be diverticulitis. In other embodiments, the physiological state is the proper functioning of the mucosal barrier, including its immune cell population. In other embodiments, the physiological state is the improper functioning of the mucosal barrier. In other embodiments, the physiological state is a disruption in the proper functioning of the mucosal barrier.

Methods for determining the physiological state may be determined by methods known in the art. For example, malnutrition may be determined by testing for amino acid, vitamin or mineral deficiencies, examining physical symptoms (e.g. edema, wasting, liver enlargement, hypoalbuminaemia, steatosis, and possibly depigmentation of skin and hair), measuring subcutaneous fat, determining stunting (%) height for age, wasting (%) weight for height and/or % of desired body weight for age and sex, or any other method known in the art. Obesity may be determined by measuring percentage body fat, total body fat, BMI, fat distribution (e.g. waist-hip ratio), or any other method know in the art. Physiological states influenced by the diet may be determined by documenting a subject's diet, physical presentation, height, weight, blood work, microbiota or a combination thereof. Methods for determining other physiological states are known in the art.

### B. Control

As used herein, the term “control” refers to one or more subjects with a physiological state different than a subject's physiological state. For example, if a subject has a pathological physiological state, a control may have a normal physiological state (i.e. be in good health). Alternatively, a subject may have a normal physiological condition (i.e. good health with no outward signs of disease) and a control may have a different desired physiological state. A skilled artisan will be able to identify an appropriate control. In some embodiments, a subject's physiological state is malnutrition and a control's physiological state is normal. In other embodiments, a subject's physiological state is obesity and a control's physiological state is normal. In other embodiments, the subject's physiological state is increased adiposity and the control's physiological state is increased adiposity. In other embodiments, the subject's physiological state is normal and the control's physiological state is increased adiposity. In other embodiments, the subject's physiological state is normal and the control's physiological state is improved digestion. In other embodiments, the subject's physiological state is normal and the control's physiological state is decreased flatulance.

By practicing the methods of the invention and comparing the taxa targeted by the immune system of a subject with a physiological state to the taxa targeted by the immune system of a control, a skilled artisan can identify taxa unique to any physiological state in a subject. When coupled to repeated measures in more than one subject with the same physiological state, a skilled artisan is able to identify taxa unique to a physiological state that is not subject-dependent (i.e. common to most or all subjects with a physiological state).

### C. Reference

As used herein, the term “reference” refers to a subject with a known physiological state and for whom the taxa that are enriched in the detection agent bound population is known. Stated another way, it is known for any given reference (i) the physiological state of the reference subject, and (ii) the taxa targeted by the reference subject's immune system. The reference may or may not be the same species as the subject. In a preferred embodiment, the reference is the same species as the subject. A reference may be a single subject or may be more than one subject with the same physiological state (e.g. a reference population). In some embodiments, a reference is a single subject. In other embodiments, a reference is more than one subject.

The present application addresses the discovery that the physiological state and the taxa targeted by the reference subject's immune system may be used to classify, predict, determine or identify the physiological state or taxa targeted by the immune system of a subject that shares one of those two features with the reference and the other feature is unknown. For example, if a subject and a reference both have the same physiological state, a skilled artisan would be able to identify the taxa targeted by the immune system of the subject as similar to the taxa targeted by the immune system of the reference without having to directly make this determination according to the methods of the invention described in Section I. Alternatively, if a subject and a reference both have similar taxa targeted by the immune system, a skilled artisan would be able to identify the physiological state of the subject as the same as physiological state of the reference. As used herein, the phrase “similar taxa” refers to the degree of identity at the family, genus or species level.

In some embodiments, there may be at least 80% identity at the family level. For example, there may be at least 80, 81, 82, 83, 84, 85, 86, 87, 88, 89, 90, 91, 92, 93, 94, 95, 96, 97, 98, 99, or 100% identity at the family level. In other embodiments, there may be 80-85% identity at the family level. In still other embodiments, there may be 85-90% identity at the family level. In yet other embodiments, there may be 90-95% identity at the family level. In additional embodiments, there may be 95-100% identity at the family level. In alternative embodiments, there may be 90-100% identity at the family level.

In some embodiments, there may be at least 70% identity at the genus level. For example, there may be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity at the genus level. In other embodiments, there may be 70-75% identity at the genus level. In still other embodiments, there may be 75-80% identity at the genus level. In yet other embodiments, there may be 80-85% identity at the genus level. In additional embodiments, there may be 85-90% identity at the genus level. In other embodiments, there may be 90-95% identity at the genus level. In other embodiments, there may be 95-100% identity at the genus level. In alternative embodiments, there may be 70-100% identity at the genus level. In different embodiments, there may be 70-90% identity at the genus level. In different embodiments, there may be 80-100% identity at the genus level.

In some embodiments, there may be at least 70% identity at the species level. For example, there may be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity at the species level. In other embodiments, there may be 70-75% identity at the species level. In still other embodiments, there may be 75-80% identity at the species level. In yet other embodiments, there may be 80-85% identity at the species level. In additional embodiments, there may be 85-90% identity at the species level. In other embodiments, there may be 90-95% identity at the species level. In other embodiments, there may be 95-100% identity at the species level. In alternative embodiments, there may be 70-100% identity at the species level. In different embodiments, there may be 70-90% identity at the species level. In different embodiments, there may be 80-100% identity at the species level.

The phrase “similar taxa” may also refer to a subset of microorganisms at the family, genus or species level rather than an entire population of microorganisms. For example, it may be more predictive to focus on the presence or absence of a particular subset of microorganisms after it has been determined that either the presence or absence of those microorganisms indicates a physiological state. In some embodiments, the subset may one or more microorganisms. For example, the subset may be at least 1, 2, 3, 4, 5, 6, 7, 8, 9, 10, 11, 12, 13, 14, 15, 16, 17, 18, 19, or 20 microorganisms. In other embodiments, the subset may two or more microorganisms. In still other embodiments, the subset may three or more microorganisms. In yet other embodiments, the subset may four or more microorganisms. In yet other embodiments, the subset may five or more microorganisms. In additional embodiments, the subset may be ten or more microorganisms. In alternative embodiments, the subset may be twenty or more microorganisms. In each of the above embodiments, the microorganism may be identified at the family, genus of species level.

### D. Other Aspects

In another aspect, the methods of the invention may further comprise characterizing the properties of the enriched taxa that are only associated with the physiological state of the one or more subjects with a physiological state. The taxa may be further characterized by any method known in the art, including suitable in vitro and in vivo assays. In an exemplary embodiment, the enriched taxa may be further characterized by inoculating the viable microorganisms into a germ-free (i.e. gnotobiotic) animal.

### III. Methods of Screening for a Therapeutic Intervention Effective at Modulating the Immune Response

In an aspect, methods of the invention provide means for screening for a therapeutic intervention effective at modulating a subject's immune response to one or more taxa. Typically, the method comprises: (a) providing a plurality of therapeutic interventions; (b) administering the therapeutic interventions to a number of subjects; (c) identifying one or more taxa targeted by the immune system of the subject after administration of the therapeutic intervention to the subject, wherein the one or more taxa targeted by the immune system are identified by the methods described above in Section I, and (d) comparing the strength of enrichment for each taxon in the detection agent bound population before and after administration of the therapeutic intervention to the subject. A change in the enrichment of a taxon after administration as compared to before administration of the therapeutic intervention indicates the therapeutic intervention was effective at modulating the subject's immune response to that taxon. In exemplary embodiments, the taxa identified in step (c) are recovered in a viable form.

Methods for measuring a change in enrichment are described in Section I, as are suitable subjects. Preferably, (i) the subject is a non-human animal model of a physiologic state and the taxa targeted by the immune system in the subject are know; and (i) the number of subjects is equal to or greater than the number of therapeutic interventions. If the taxa targeted by the immune system in the subject are not known, a suitable biological sample must be obtained prior to administration of the therapeutic intervention in order to identify taxa targeted by the subject's immune system. In some animals, a subject is a laboratory animal. In a preferred embodiment, a subject is a gnotobiotic animal colonized with microbiota from one or more humans with a known physiological state.

As noted above, the term “therapeutic intervention” refers to a pharmaceutical composition or drug product comprising an API, a biologic, or a combination thereof, as well as dietary interventions. Non-limiting examples of dietary interventions may be prebiotics, probiotics, synbiotics, caloric restriction, caloric supplementation, food group restriction (e.g. lactose-free, gluten-free, soy-free, peanut-free, nut-free, or wheat-free diets), or changes in the diet that increase or decrease the amount one or more food group, or one or more nutrient and/or vitamin, relative to the total amount of food. Probiotics are live microorganisms, which when administered in adequate amounts confer a health benefit on a subject. In some embodiments, a probiotic is a single taxon. In other embodiments, a probiotic is one or more taxa. In still other embodiments, a probiotic is two or more taxa. In yet other embodiments, a probiotic is three or more taxa. In different embodiments, a probiotic is four or more taxa. In alternative embodiments, a probiotic is five or more taxa. A prebiotic is a compound that promotes one or more changes in the composition or activity of a subject's microbiota. A synbiotic is a composition comprising one or more probiotics and one or more prebiotics that results in a synergistic net health benefit.

Any therapeutic intervention known in the art may be screened to determine if it is effective at modulating the subject's immune response to one or more taxa. Also contemplated are those therapeutic interventions not yet known in the art but which may be screened according to the methods of the invention.

In some embodiments, the therapeutic intervention is selected from the group consisting of an API, a biologic, a dietary intervention, and a combination thereof. In a preferred embodiment, the therapeutic intervention is a probiotic. In another preferred embodiment, the therapeutic intervention is a prebiotic. In another preferred embodiment, the therapeutic intervention is a synbiotic.

In other embodiments, the therapeutic intervention is a composition comprising Clostridium scindens, Akkermansia muciniphila, or a combination thereof. In still other embodiments, the present application encompasses the use of a compound, a biologic, a probioitic, a prebiotic, a synbiotic, an antibiotic, a change in diet, or a combination thereof, comprising the microorganisms present in one or more taxa identified by the methods detailed above in the modulation of the immune system of the subject.

A therapeutic intervention may be formulated and administered to a subject by several different means. For instance, a composition may generally be administered orally, parenteraly, intraperitoneally, intravascularly, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In the case of the gastrointestinal tract, the preferred method of administration of the therapeutic intervention may be orally as a pill, or a solution or as an incorporated component of a dietary ingredient or ingredients. Methods known in the art could also be used to deliver the therapeutic agent to specified regions of the gut (e.g. the colon). Other methods, also known in the art, could be used to deliver the therapeutic agent to other body habitats (e.g., intravaginally).

A change in the enrichment may be an increase in enrichment or a decrease in enrichment. In some embodiments, a change may be an increase in enrichment. In other embodiments, a change may be a decrease in enrichment. The amount of a change indicates the degree of effectiveness. For example, the greater the change, the more effective the therapeutic intervention and vice versa. In some embodiments, a change in enrichment may be at least 5%. For example, a change in enrichment may be at least 5%, 10%, 15%, 20%, 25%, 30%, 35%, 40%, 45%, 50%, 55%, 60%, 65%, 70%, 75%, 80%, 85%, 90%, or 95% or more. In other embodiments, a change in enrichment may be at least 100%. For example, a change in enrichment may be at least 100%, 125%, 150%, 175%, 200%, 225%, 250%, 275%, 300% or more. In still other embodiments, a change in enrichment may be at least 400%. For example, a change in enrichment may be at least 400%, 500%, 600%, 700%, 800%, 900%, or 1000% or more.

In some embodiments, the change is a decrease in enrichment of Enterobacteriaceae. In other embodiments, the change is an increase in the enrichment of Clostridium scindens, Akkermansia muciniphila, or a combination thereof. Changes in the enrichment of these microorganisms and others, and methods for measuring their change, are described in further detail in the Examples.

### IV. Methods for Determining the Effectiveness of a Therapeutic Intervention at Modulating the Immune Response in a Subject

In an aspect, methods of the invention provide means for determining the effectiveness of a therapeutic intervention at modulating a subject's immune response to one or more taxa. Typically, the method comprises (a) identifying one or more taxa targeted by the immune system of the subject before and after administration of the therapeutic intervention to the subject, wherein the one or more taxa targeted by the immune system are identified by the methods described above in Section I, and (b) comparing the strength of enrichment for each taxon in the detection agent bound population before and after administration of the therapeutic intervention to the subject. A change in the enrichment after administration as compared to before administration of the therapeutic intervention indicates the therapeutic intervention was effective at modulating the immune response. Suitable subject are described in Section I.

Methods for measuring a change in enrichment are described in Section I, as are suitable subjects. Suitable therapeutics and changes in enrichment are described above in Section III. In some embodiments, a subject is a companion animal. In other embodiments, a subject is a livestock animal. In still other embodiments, a subject is a laboratory animal. In preferred embodiments, a subject is a human.

### EXAMPLES

The following examples are simply intended to further illustrate and explain the present invention. The invention, therefore, should not be limited to any of the details in these examples. All references cited herein are incorporated by reference in their entirety.

### Example 1

### Methods of Isolating Microorganisms Targeted by a Host's Immune System

Members of the human gut microbiota typically have mutually beneficial relationships with their hosts. The host maintains these relationships in part through the production of antibodies, such as IgA, by mucosal immune cells. These antibody: microorganism interactions serve to exclude microbial epitopes so as to avoid untoward immune responses to these organisms. As such, these antibody responses form an integral part of the intestine's mucosal barrier. Breakdown of the intestine's mucosal barrier can activate unwanted immune responses to normally beneficial microbes leading to diseases within and outside of the gut. Antibody responses to members of the gut microbiota provide a way of tagging these microbes, in healthy as well as in disease states, since antibodies bind to the surface of these microorganisms. Thus, these antibody responses provide a way of identifying organisms, distributed along the length and width of the gastrointestinal tract, that are recognized by, and are the targets of, the immune systems of individuals representing various ages, geographic locations, cultural traditions and life styles, diets, physiological states, and disease states. These microorganisms, their spatial distribution along the length and width of the gut, and their functional interactions with components of the immune system, may serve critical roles in promoting health within and/or outside of the gut, or may be important agents of disease. Therefore, identifying gut microbes that are that targets of host immune responses may have important diagnostic and therapeutic value.

Microorganisms targeted by a host's immune system were isolated and characterized using the following steps: (1) preparation and fluorescent activated cell sorting of (gut) microbes that are the targets of host immune responses in a manner that preserves their viability; (2) ex vivo characterization of the sorted fractions; (3) transplantation of sorted microbes into and propagation within gnotobiotic animals, (4) in vivo characterization of the organisms, and (5) retrieval of the sorted and transplanted organisms from gnotobiotic animal recipients for further ex vivo characterization.

Step 1—Preparation and FACS of Gut Microbes that are the Targets of Host Immune Responses in a Manner that Preserves their Viability.

All or a portion of a freshly obtained or previously frozen sample of a human microbial community harvested from its body habitat (e.g. feces) was homogenized in a sterile buffered solution. For example, ˜10-50 milligrams of feces are typically added to 1 ml of sterile phosphate buffered saline (PBS) and mixed by vortexing for 5 min at room temperature. Fecal samples can be obtained from human subjects directly or from mice harboring a transplanted intact (uncultured) human gut microbial community, or in yet another embodiment, from gnotobiotic mice harboring a transplanted microbial community consisting of microorganisms isolated on the basis of their association with components of the immune system. Care was taken to avoid overly vigorous disruption of fecal material to preserve the integrity of the antibody-bacteria complexes. After the fecal material was broken into small granules, the homogenate was placed on ice for ˜5-10 min to permit settling (by gravity) of larger particulate matter and its separation from more buoyant bacteria. Next, 200 microliters of the cleared supernatant were then filtered through 70-micron pore diameter sterile nylon filters into a new container. The sample was then centrifuged briefly (at approximately 10,000×g) and the resulting pellet, containing primarily bacteria and bound immunoglobulin, was washed once with sterile PBS.

The pellet was subsequently resuspended in 100 microliters of PBS solution containing a 1:50 dilution of polyclonal goat anti-IgA antibody conjugated to the fluorescent molecule Dylight 649 (AbCam PLC; similar to the fluorescent molecule allophycocyanin). After incubating on ice for 30 min, bacteria were pelleted, washed with 1 ml of PBS, and then resuspended in a solution containing 0.9% NaCl (w/v), 0.1 M HEPES and a 1:4000 dilution of SytoBC, a commercially available fluorescent DNA dye (Molecular Probes) that has spectral properties similar to fluorescein isothiocyanate (FITC).

Once stained with both the anti-IgA antibody and the DNA-directed dye, bacteria were analyzed and sorted using FACS. Several parameters were utilized to reliably identify antibody (e.g., IgA)-bound bacteria (“gating”): (i) particles with bacteria-like size were identified by using the Forward Scatter (FSC) and Side Scatter (SSC) channels; (ii) particles of the appropriate size were selected that were also bound to SytoBC indicating the presence of DNA (Use of the DNA stain is an important step in this protocol, as it allows bacteria (which have DNA) to be distinguished from auto-fluorescent material that would otherwise interfere with the detection of the presence of bacteria with bound antibodies); and, (iii) particles of the appropriate size and containing DNA were assessed for the presence or absence of bound host antibodies (e.g., IgA) by quantifying the strength of the Dylight 649 labeled anti-antibody. The relative proportions of Ig (e.g. IgA) bound versus unbound bacteria were quantified at this step.

Three populations of bacteria were collected by FACS for subsequent analysis. The first population was selected purely on the basis of size and was representative of all bacteria present in the fecal sample (the “input” population). The second population was comprised of bacteria that have stained positive for the presence of DNA, but negative for host Ig (e.g., the “IgA negative population”). The third population stained for the presence of DNA and host Ig (e.g., the “IgA positive population”).

Note that these procedures can be applied to a human sample directly, or fecal samples collected from gnotobiotic animals with various genetic backgrounds that were the recipients of a transplanted intact uncultured, human gut microbial community sample, or gnotobiotic animals that were the recipients of a transplanted culture collection generated from the human gut microbial community sample. These gnotobiotic animals can be mice, or pigs. They can be fed a variety of diets resembling those of the human microbiota donor or synthetic diets with systematically varied ingredients. The microbial community can be derived from a given body habitat (e.g. the gut) of a human or from a given body habitat of non-human species.

Step 2—Ex Vivo Characterization of the Sorted Fractions.

The three different populations of bacteria obtained from Step 1, all derived from a single fecal specimen, were used to identify bacteria that contain bound host antibodies (e.g. IgA) using culture-independent methods: namely sequencing the phylogenetic marker gene, 16S rDNA). Methods for multiplex pyrosequencing of PCR amplicons generated from selected variable regions of the bacterial 16S rRNA genes are well known in the art (See, Turnbaugh et al., 2009; Goodman et al., 2011). A small aliquot of bacteria from each sorted population was used to perform 16S rRNA PCR using sample specific error correcting barcodes attached to primers that are targeted to conserved regions of the bacterial 16S rRNA gene that flank a targeted variable region (e.g., V2, or V4). Barcoded amplicons generated by PCR were pooled and subjected to sequencing using a highly parallel DNA sequencer. Sequence data was then analyzed using publicly available software to obtain a taxonomic description of the make-up of each of the three populations (e.g., the input, Ig-negative and Ig-positive populations).

These taxonomic descriptions were used to calculate a normalized value for the strength of Ig binding in a variety of ways. Generally speaking, within a single sample, a given taxon is more likely contained in the bound Ig (e.g., IgA) population if its proportional makeup within the host Ig-positive population is greater than in the Ig-negative population. Second, with multiple replicate samples available, a paired Student's t test may be applied comparing the proportional make up of taxa in the Ig-positive population to the proportional make up in the IgA negative population (paired by sample). Such an approach may be used to ascertain the statistical likelihood that a given taxon is bound to Ig over a population of samples or repeated measurement of the same sample. Additionally, by collecting the unmanipulated input population, a normalized value for the strength of Ig binding may be calculated. In certain embodiments, a normalized value for the strength of Ig binding may be calculated using the equations described in Section I. This normalized value may be used to compare the strength of an Ig response to a given taxon within and across different types of samples.

These analyses were also coupled with efforts to culture components of the sorted anaerobic or more aerotolerant bacterial species present in the various sorted populations. The IgA positive bacterial population was cultured directly after sorting (using both anaerobic and aerobic methods) or introduced into germ-free mice for further characterization. Sorted and cultured bacterial populations were further characterized, including analysis of their genome sequences, their growth properties in the presence or absence of various nutrients, their transcriptional and metabolic responses to these nutrients, their sensitivity or resistance to previously discovered or newly discovered antibiotics, and their ability to produce molecules with biological activities against other microbes and/or host cell populations.

Step 3—Transplantation of Sorted Microbes into and Propagation within Gnotobiotic Animals.

To optimize recovery of live bacteria, all preparation steps described above were performed within an anaerobic chamber and 0.1% cysteine is added to all buffers. Sorted fractions, notably the antibody-positive fraction were introduced into recipient gnotobiotic mice by gavage using methods described in Goodman et al (2011). Recipient mice varied in terms of their age, gender or genetic background. Animals were fed a variety of diets including those resembling those of the human donor. These diets can be sufficient or deficient in macro or micronutrients. They can be synthetic, having systematically varied concentrations of macro or micronutrients. Diets were sterilized by irradiation or autoclaving prior to administration.

Mice can not only be gavaged with one of the three sorted populations described above, but also with various combinations of populations from a single donor, or a mixture of comparable populations from several donors, including donors with different phenotypes (e.g. IgA-positive populations generated from the fecal microbiota of a healthy and a malnourished co-twin in a discordant twin pair).

A given sorted population can be supplemented with other designated microbial species or microbial consortia to determine the effects of these species or consortia on the properties that are conveyed to the recipient mice by the sorted population. Such effects may be used to enhance or attenuate the properties of the sorted population, including those conveyed to the host gnotobiotic animal.

A given sorted population can be from fecal samples obtained from a mouse that had previously been colonized with a sorted sample derived directly from a human specimen and fed one of several different diets. In these cases, the sorted population would be generated from the mouse fecal sample using antibodies directed against mouse Ig (e.g. rather than using a labeled anti-human IgA, an anti-mouse IgA would be employed). Note that recipients of the sorted populations generated from the fecal microbiota of these mice may receive the same diet as the donor mouse or different diets to ascertain the interactions between diet and the sorted and transplanted microbial populations.

Step 4—In Vivo Characterization of the Sorted Microbial Populations.

Recipient animals are maintained in gnotobiotic isolators and are followed over time, with periodic sampling of their feces, urine, and blood, and with periodic measurements of various physiologic parameters, including weight, food consumption, nutritional status/body composition (by quantitative magnetic resonance imaging), metabolic rate (by open circuit indirect calorimetry), metabolic phenotypes (by mass spectroscopic or NMR analyses of their biofluids such as urine or blood or other types of biospecimens such as feces), immune phenotypes (including gut barrier functions and responses to vaccination), and behavior.

Fecal samples can be used to define the organismal and gene composition of the gut microbiota of recipient gnotobiotic mice (e.g. by sequencing amplicons generated from bacterial 16S rRNA genes and by shotgun sequencing of community DNA). Microbiome gene expression can be characterized by quantifying mRNA (using microbial RNA-Seq), protein (with mass spec-based proteomics) and/or metabolites (by NMR or mass spectrometry) in gut contents (including feces). Microbial and host co-metabolism can be ascertained by profiling metabolites in intestinal contents, blood and urine collected from recipient animals.

Step 5—Retrieval of the Sorted and Transplanted Organisms from Gnotobiotic Animal Recipients for Further Ex Vivo Characterization.

See steps 2-4 above. Note that multiple rounds of sorting and transplantation can occur to further purify taxa that are the targets of host immune responses. After each round, the fecal sample can be sorted and the sorted populations transplanted directly into the next round of gnotobiotic mice or the sorted population could be cultured prior to transplantation.

### Example 2

### Solving Methodologic Challenges and Calculating Sorting Efficiency

Contamination of the FACS Machine by Bacteria—

FACS machines are used primarily for sorting eukaryotic cells (FACS sorters) and have a complex fluidics system that, depending on their design, can become contaminated with environmental bacteria. Early experiments demonstrated that these bacteria can be detected by 16S rRNA sequencing, even after sterilization of all the associated fluids. This problem was addressed in three different ways. First, an existing FACS sorter designed to minimize contamination was used (e.g. a FACS Aria III where there is a minimization of areas within the machine where bacteria can become trapped). Second, the machine was prepared for a day of sorting by sterilizing the FACS fluidic system using a manufacturer recommended protocol. Third, “pre-sort” and “post-sort” control samples from the FACS machine were collected to track potential contamination of the machine and cross-contamination over the course of an experiment. If these control samples demonstrate a significant amount of contamination, samples collected that day can be corrected for this contamination by removal of contaminating taxa from the data analysis.

False Positives in the Sorted IgA Positive Population—

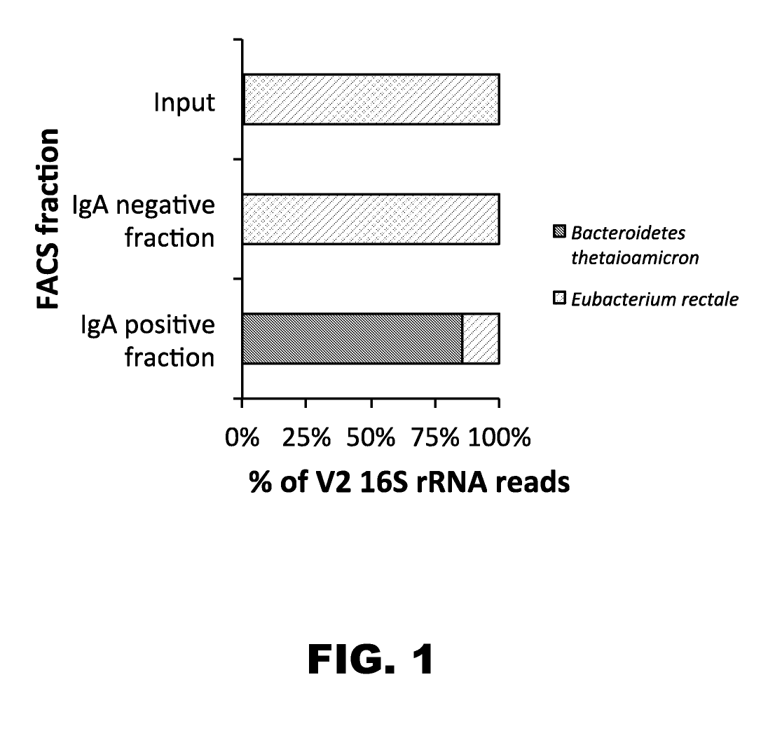

FACS machines are primarily used to distinguish and separate mammalian cells, which are many-fold larger than most bacteria. Furthermore, the degree to which a commercially available FACS machine is able to purify a given bacteria based on its binding to IgA is unknown. To address this issue, a monoclonal IgA antibody to Bacteroides thetaiotamicron was used (MAb 225.4, Peterson et al., 2007) to show that MAb 225.4-bound B. thetaiotaomicronfrom was enriched from an ˜0.1% of a mixed input population to 80% of the IgA positive population (as measured by 16S rRNA; see FIG. 1).

While these experiments demonstrate that FACS can selectively enrich for a specific taxon based on the presence of a specific IgA antibody, it also shows that the purity of an IgA positive bacterial population will be significantly less than what can be achieved when enriching mammalian cells (which often exceeds 99% purity). The consequence of this observation is that determining which bacteria are “truly” bound to IgA is more complicated than simply determining the identity of bacteria comprising the IgA positive pool because a substantial proportion of bacteria within the IgA positive pool may be false positives.

The protocol described above is able to overcome this challenge by simultaneously collecting an IgA negative and an IgA-positive population. Enrichment can be determined by comparing the composition of the two populations and noting those taxa whose representation are greater in the IgA positive population. When coupled to repeated measures (either of the same sample or over a population), a p value can be generated that indicates the degree of confidence for that taxa being significantly enriched in the IgA positive population.

Additionally, non-specific binding of the secondary (anti-IgA) antibody was considered as a potential source of false positive taxa. As a result, a control sample from Rag1−/− mice that lack B-cells and are unable to produce antibody was used to assay for specificity of binding. Alternatively, isotype control antibodies have also been used when targeting human IgA.

Compare IgA Positive Taxa Across Samples—

As discussed above, comparing the taxonomic composition of the IgA positive and IgA negative populations defines bacteria with bound IgA through correction of false positives. Using the model system described above, it was been demonstrated that with an additional piece of data (the composition of the “input population”), there is a linear relationship (for a given taxon) between IgA positive, IgA negative and input populations. The slope of this line, with intercept equal to zero, can be calculated by:

$$\frac{log ( {IgApositive}_{taxon} / {IgAnegative}_{taxon} )}{- log ( {Input}_{taxon} )}$$

This number represents the strength of enrichment of a taxon in the IgA positive fraction with any value greater than 0 representing enrichment in the IgA positive population. The strength of enrichment, in turn, is determined by multiple factors including the amount of IgA present, the strength of IgA binding, and factors related to the efficiency of FACS sorting, but is not dependent on the abundance of the taxa within the sample, allowing cross-sample comparisons.

### Example 3

### Exemplary Applications of the Invention

Using a “humanized” gnotobiotic mouse model of malnutrition, a consortium of bacteria capable of causing disease and taxa with protective, disease-mitigating properties has been identified. Reconstituting a human intestinal microbial community within previously germ-free mice was used to study the role of the gut microbiota in twins discordant for kwashiorkor, a severe form of childhood malnutrition. Mice received fecal microbiota transplants from a twin pair where the co-twins were discordant for kwashiorkor. Mice that received the kwashiorkor co-twin's microbiota and were fed a micro- and macronutrient deficient diet representative of the diet consumed by the microbiota donor, develop more weight loss than mice fed the same diet but that had received a fecal microbiota transplant from the healthy co-twin.

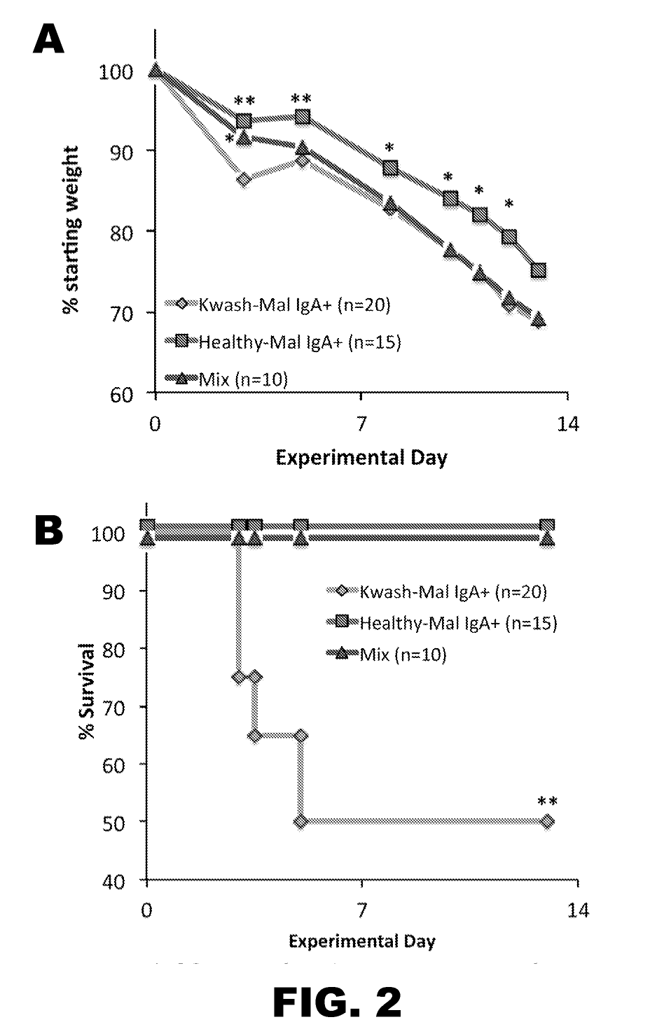

When the IgA positive fraction of bacteria was isolated from the fecal microbiota of kwashiorkor microbiota transplant recipients fed a Malawi diet, and introduced into another generation of germ-free mice who were fed the same nutrient deficient Malawi diet, these mice experienced rapid decreases in body weight and death in contrast to mice receiving the IgA positive bacterial fraction from mice harboring the healthy co-twins microbiota (FIGS. 2A and 2B). Furthermore, if the IgA positive fraction from both the kwashiorkor and healthy group were mixed prior to introduction into germ-free mice, the recipient animals exhibited significantly less weight loss and mortality, implying the presence of a protective taxon or taxa within the sorted IgA positive population obtained from the mouse with the healthy co-twin's microbiota. Using a combination of fecal bacterial community profiling (16S rRNA) and BugFACS several species were identified, including Akkermansia muciniphilia as well as Clostridium scindens as potential candidates mediating these protective effects.

### Example 4

### Human Application of the Methods of the Invention

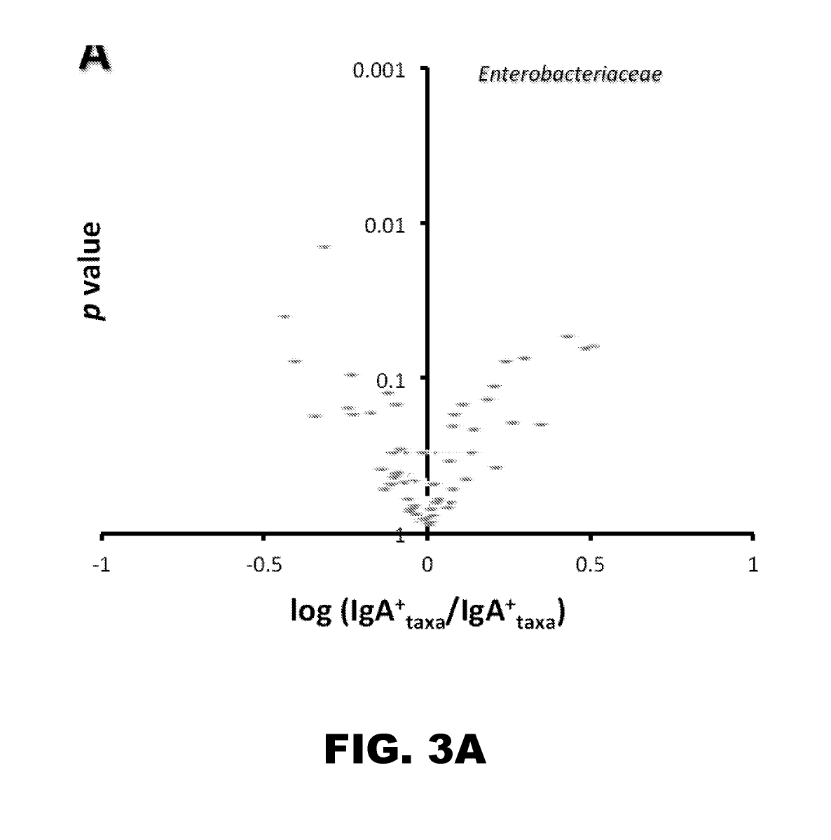

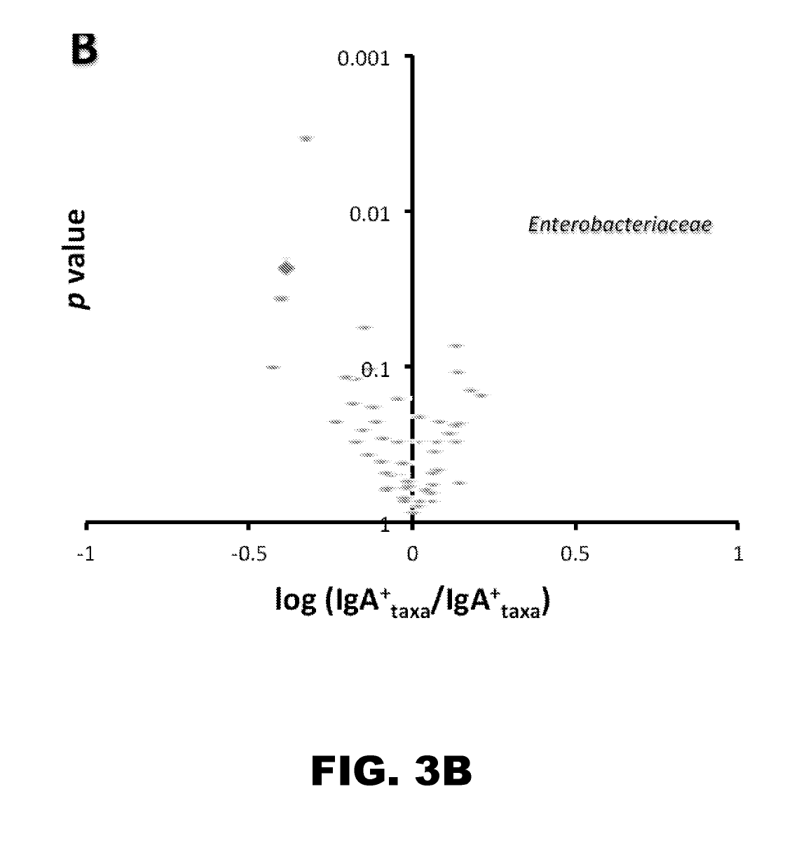

Fecal samples obtained from human twins discordant for kwashiorkor were analyzed using the methods described in Examples 1-3. In particular, BugFACS was directly applied to the human samples (using an anti-human IgA antibody) and, aside from additional handling precautions, no additional modifications were made to the protocol (FIG. 3).

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

### Example 5

### Rationale for Bug FACS and Validation of BugFACS Protocols

To determine which members of the intestinal microbiota are targeted by the host's muocsal immune system, mucosal immunoglobulin A (IgA) was used to identify bacterial taxa that had stimulated an antibody response. IgA is a major component of the mucosal immune response that aids in protecting and maintaining barrier function at mucosal surfaces. As a component of the adaptive immune response, IgA is produced by B cell/plasma cells and is actively transported across mucosal epithelial surfaces into the sinuses, airways, and, in particular, into the lumen of the gastrointestinal tract where an estimated eight grams of IgA is produced by an individual on a daily basis. IgA functions by binding bacterial, food and other antigens to sequester them away from the mucosal surface and prevent direct interaction with the host, a principle known as “immune exclusion”. Published reports have demonstrated that Fluorescence Assisted Cell Sorting (FACS) can be used to quantify the proportion of fecal bacteria that are coated in IgA (Kawamoto et al. 2012, Hapfelmeier et al. 2010), however no attempt was made to collect or manipulate viable organisms. FACS has also been used to sort bacteria labeled with DNA-specific dyes (Maurice et al, 2013). We developed a method for examining diet-by-microbiota-mucosal immune system interactions using FACS and for examining the biological significance of IgA-targeted gut bacteria by transplantation of FACS-purified fractions into germ-free mouse recipients.

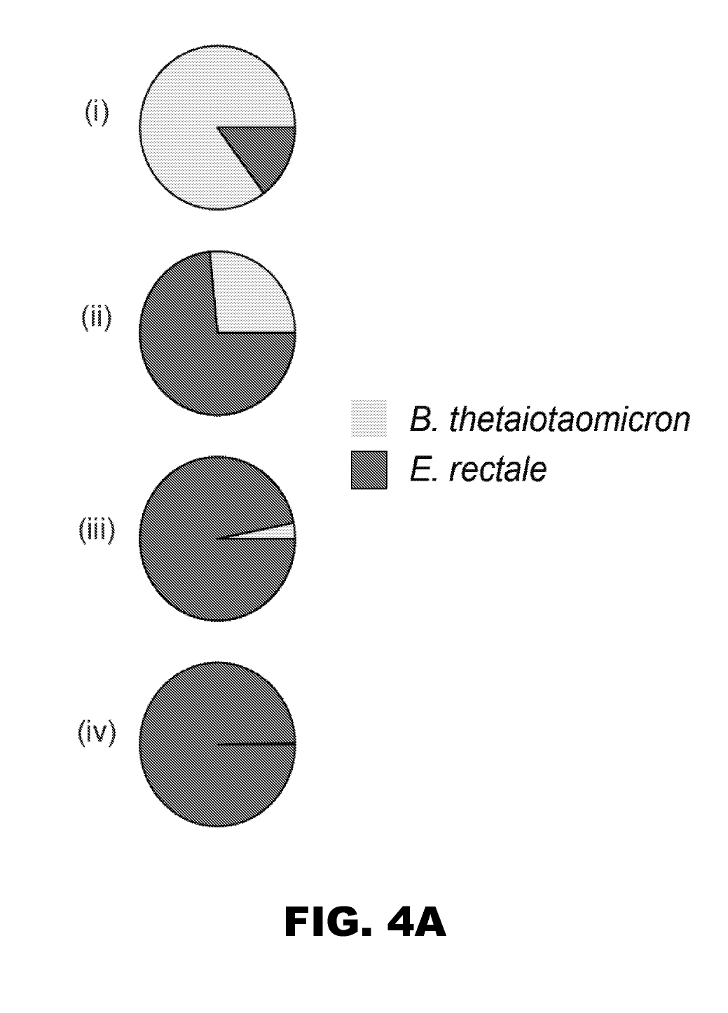

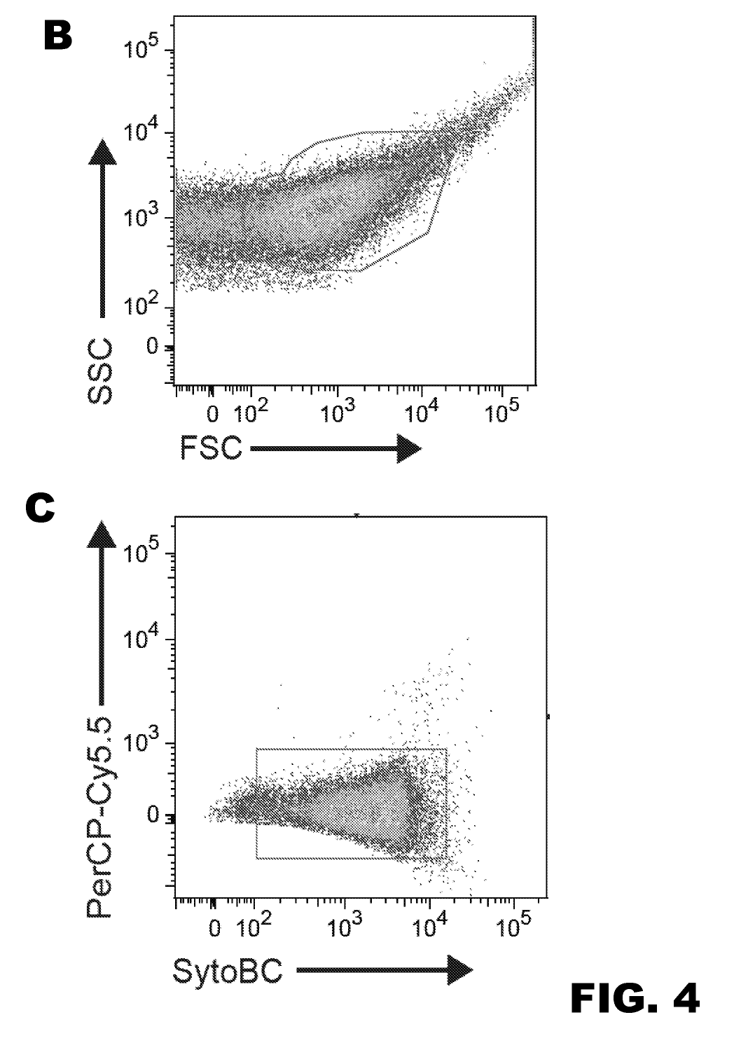

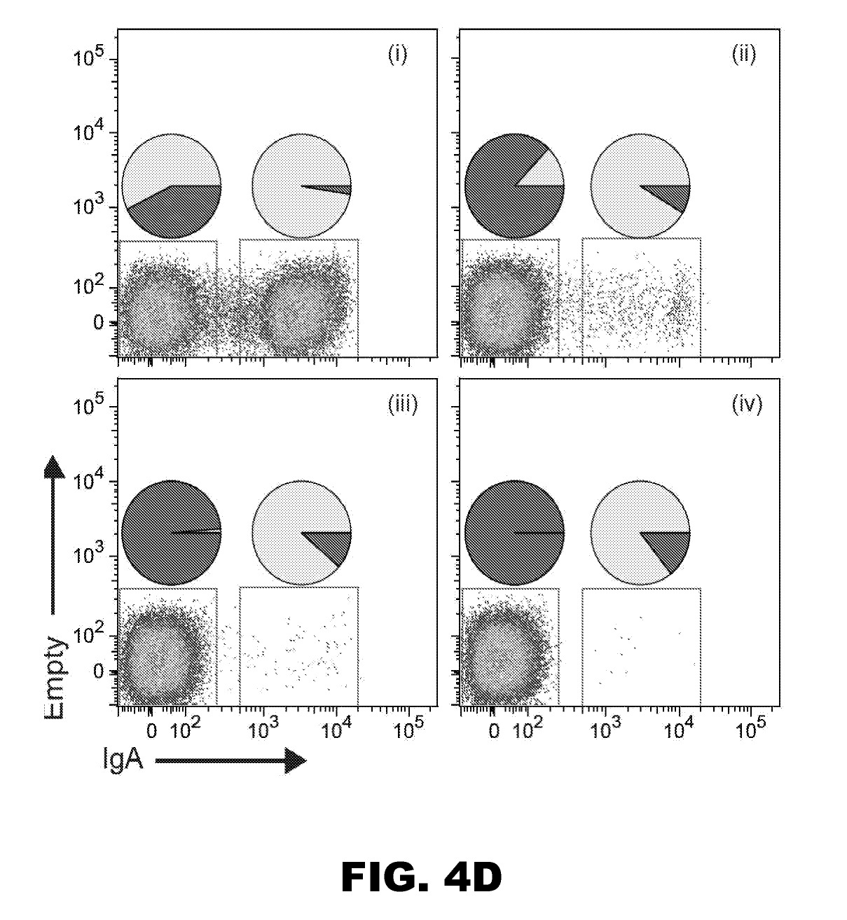

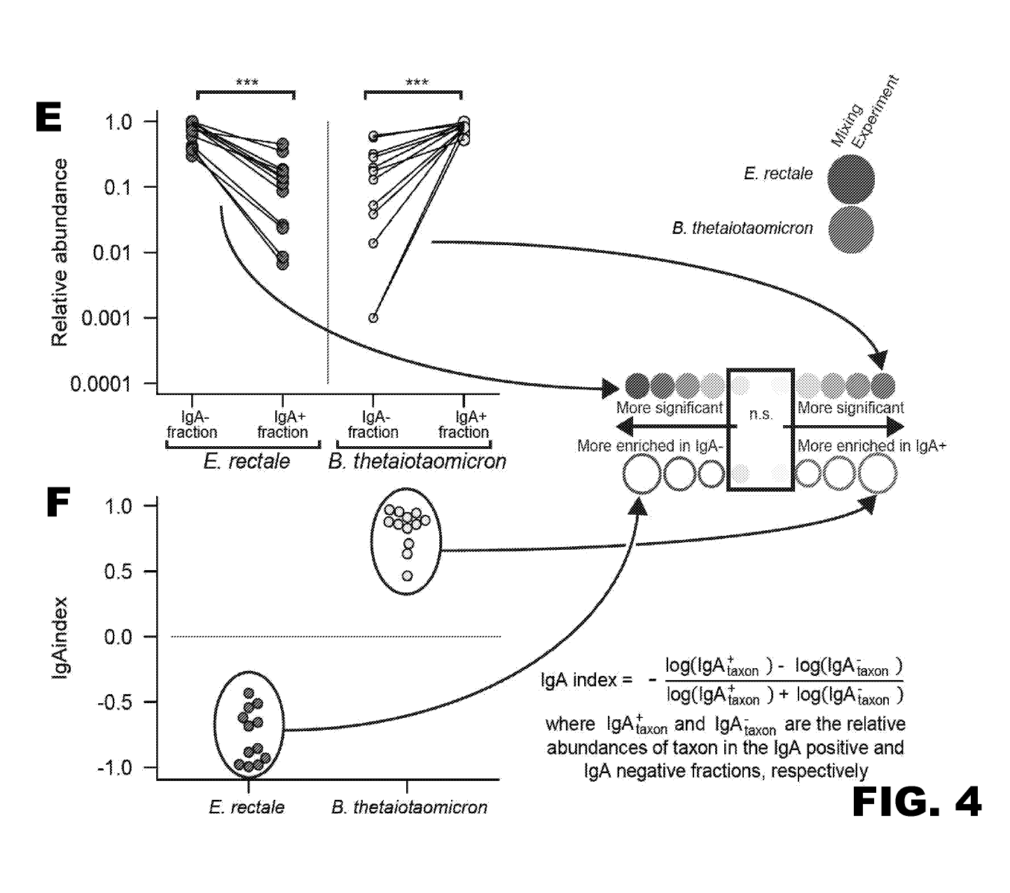

In order to validate that the protocol could be used to identify IgA targeted microbes in both in vitro and in vivo systems, a previously generated monoclonal IgA antibody (Peterson et al., 2007) was used to demonstrate that Bacteroides thetaiotamicron could be reproducibly enriched from a mixture of B. thetaiotamicron and Eubacterium rectale as measured by V2-16S rRNA sequencing (FIG. 4). Second, in a mouse transgenic for a T-cell receptor with reactivity to members of the genus Bacteroides, it was also shown that there is significant enrichment of IgA coated Bacteroides relative to non-trangenic mice from the same genetic background.

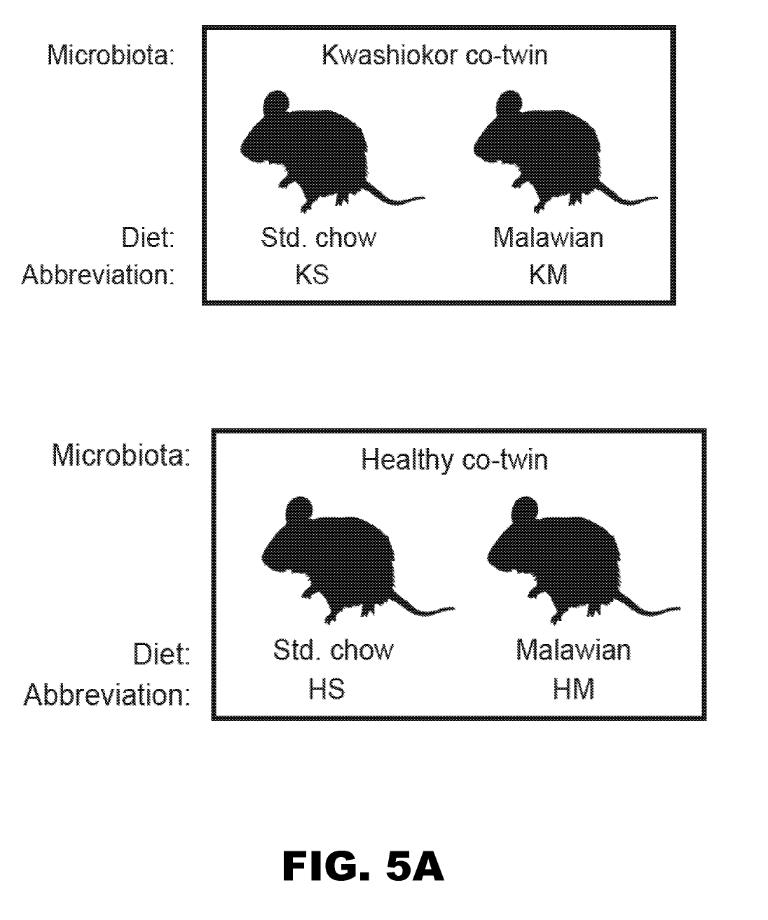

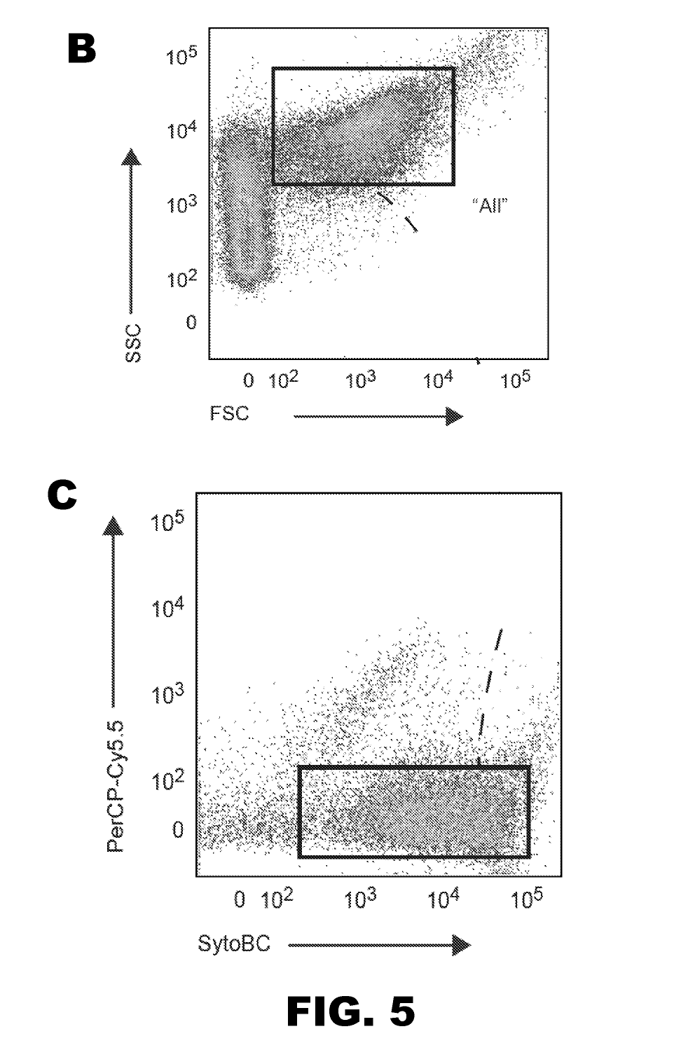

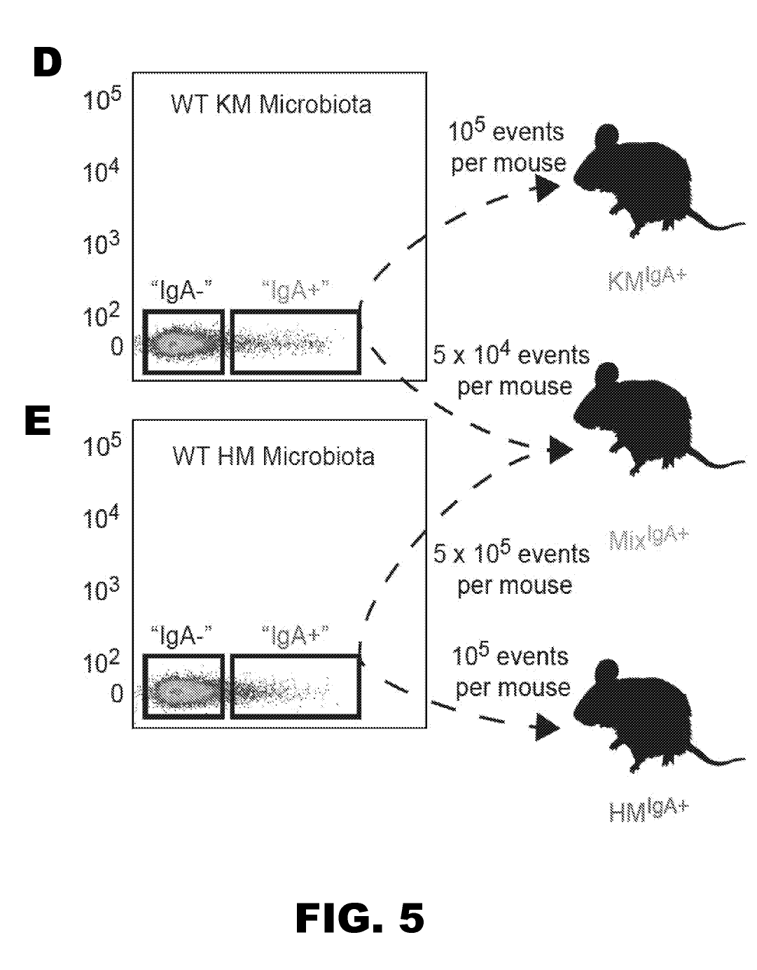

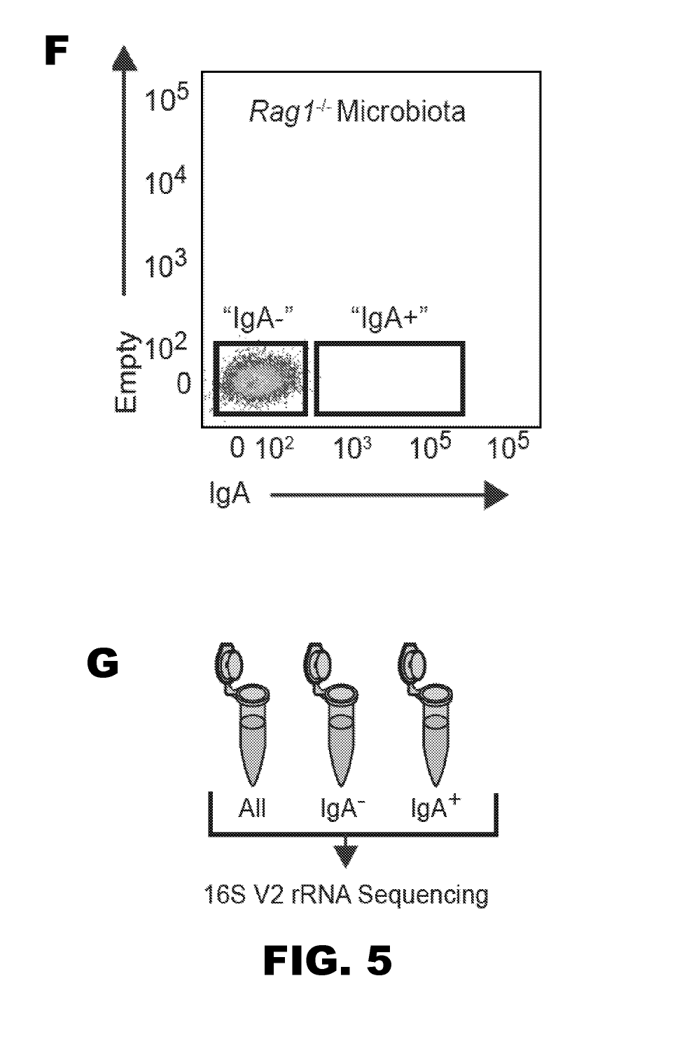

This procedure (FIG. 5B-D, FIG. 4), known as BugFACS, can be followed by 16S rRNA sequencing of the sorted fractions to identify the intestinal microbial targets of the intestinal IgA response (FIG. 5D, i.e. analytical BugFACS) or, by incorporating standard anaerobic techniques to minimize oxygen exposure, can be used to recover viable consortia of bacteria that are enriched for taxa that are targets of an IgA response. These consortia can be inoculated into germ free mice in a way that is functionally analogous to adoptive transfer (FIG. 5C, i.e. microbial adoptive transfer).

### Example 6

### Applying BugFACS to Assay the Microbial Targets of Gut Mucosal IgA Responses in Mice Harboring Transplanted Fecal Microbiota from Twins Discordant for a Form of Severe Acute Malnutrition (Kwashiorkor)

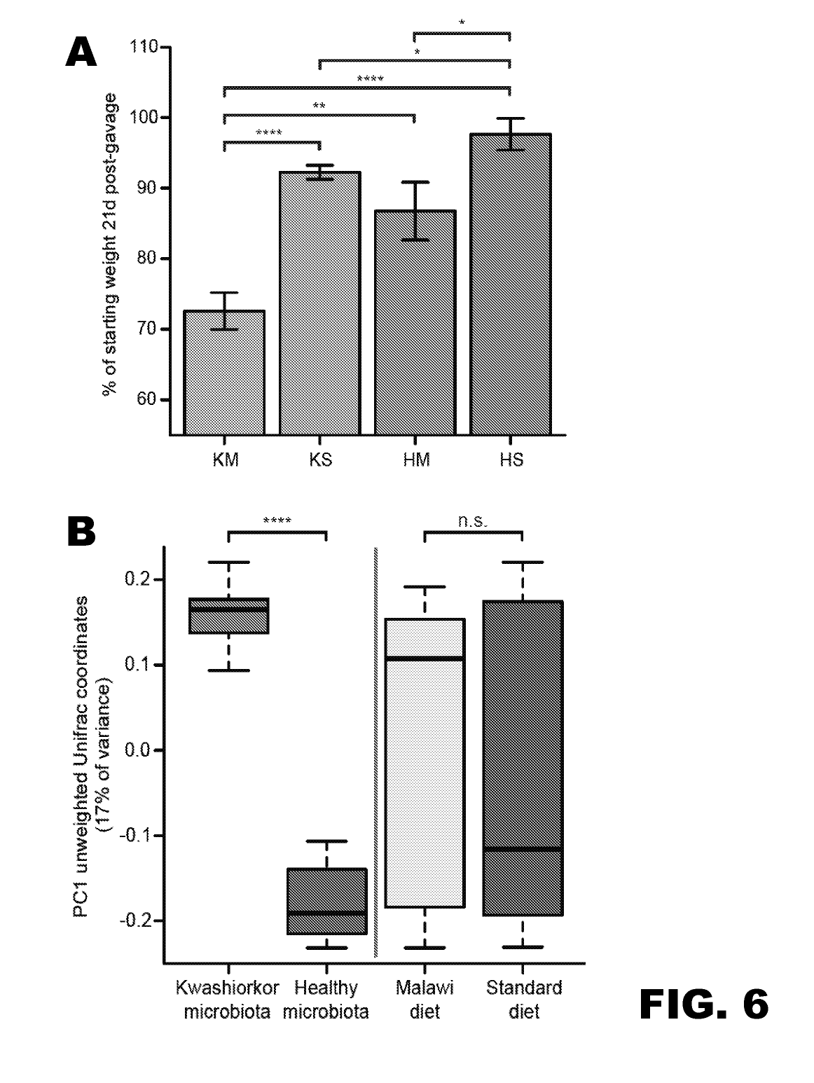

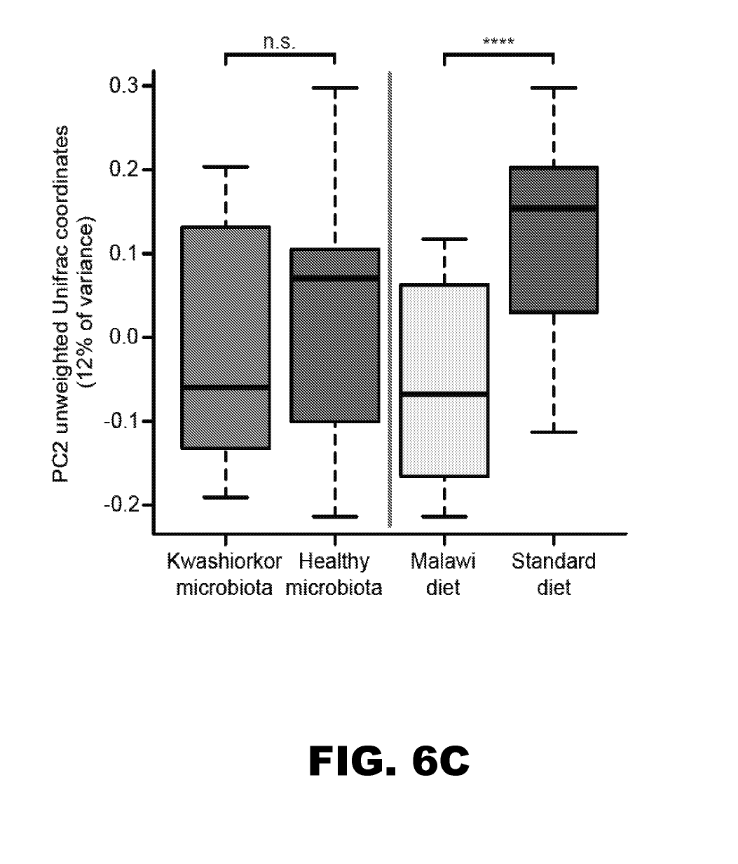

As part of ongoing efforts to better understand how the microbiota participates in the development of malnutrition, a humanized mouse model was recently described in which mice were colonized with a microbiota from twin pairs discordant for kwashiorkor, a form of severe acute malnutrition (Smith/Yatsunenko et al., 2013). These mice were then fed a macro- and micro-nutrient deficient Malawi diet or a macro- and micro-nutrient sufficient mouse chow (‘standard diet’, which is low in fat and rich in plant polysaccharides) (FIG. 5A). Mice humanized with the microbiota from the co-twin with kwashiorkor and fed the Malawian diet (KM mice) lost significantly more weight when compared to mice fed the same diet but humanized with the microbiota from the healthy co-twin (HM mice, FIG. 6). Mice that were fed the standard diet lost less weight than counterparts fed the deficient Malawi diet, regardless of the microbiota (KS and HS mice respectively.)

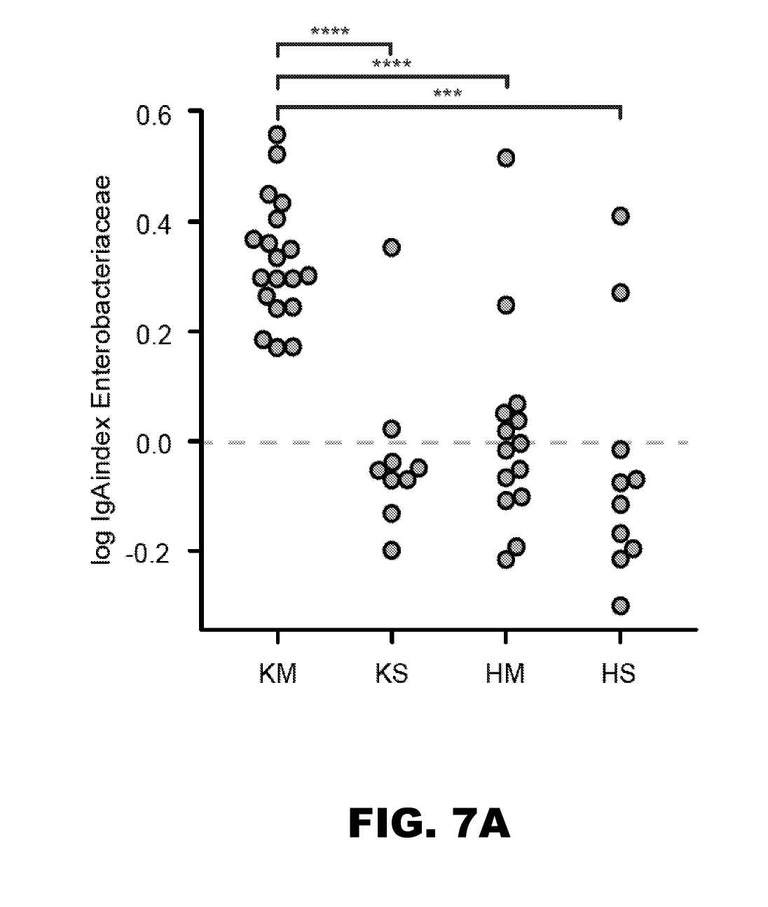

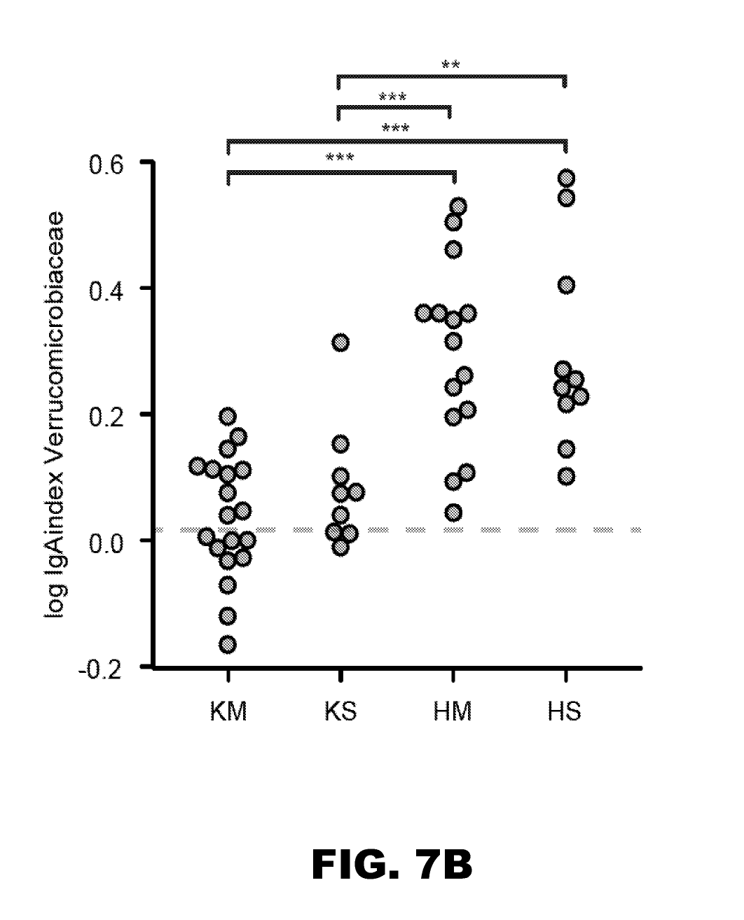

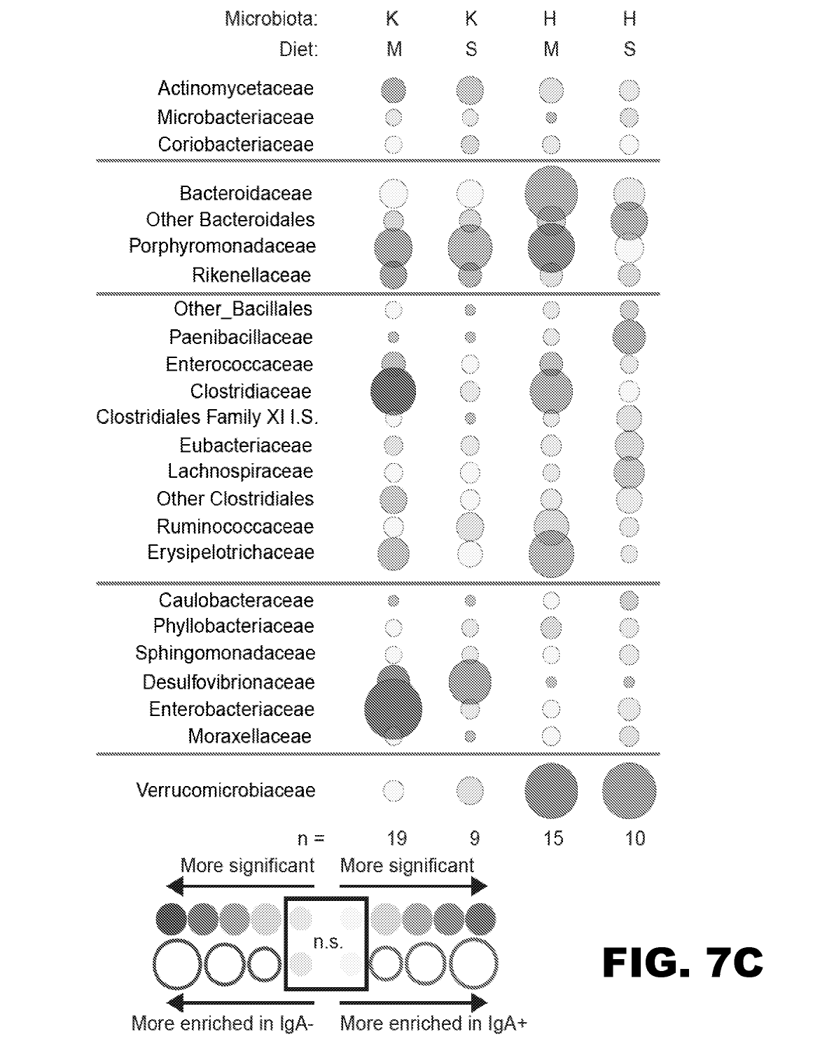

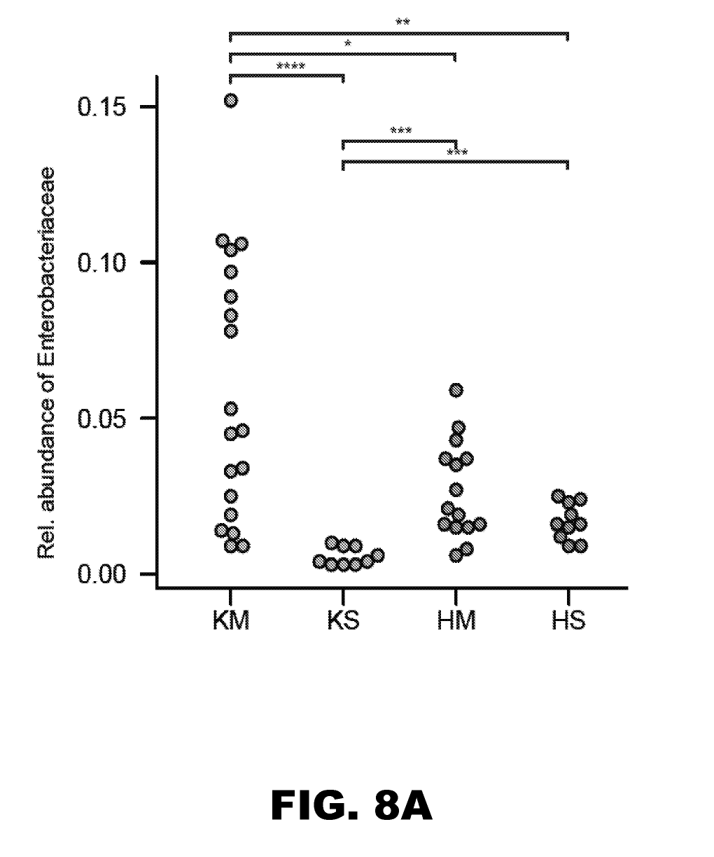

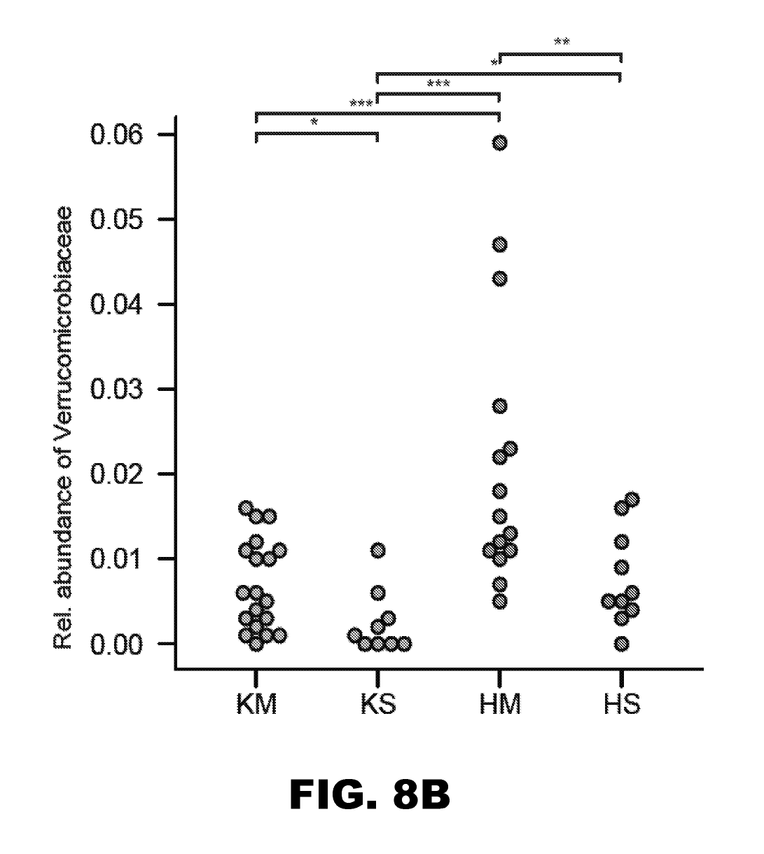

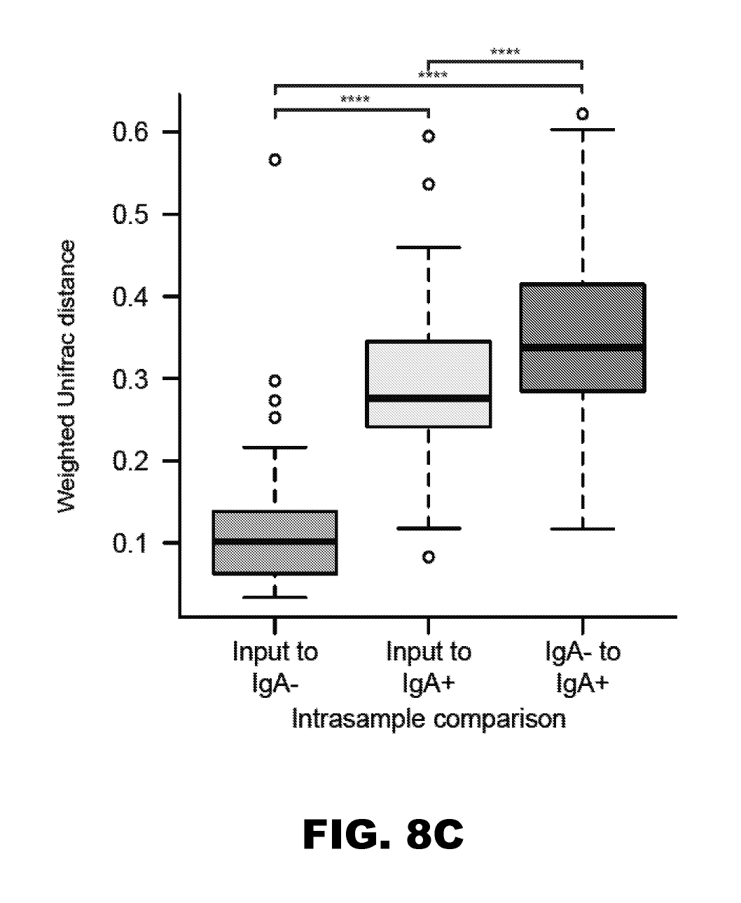

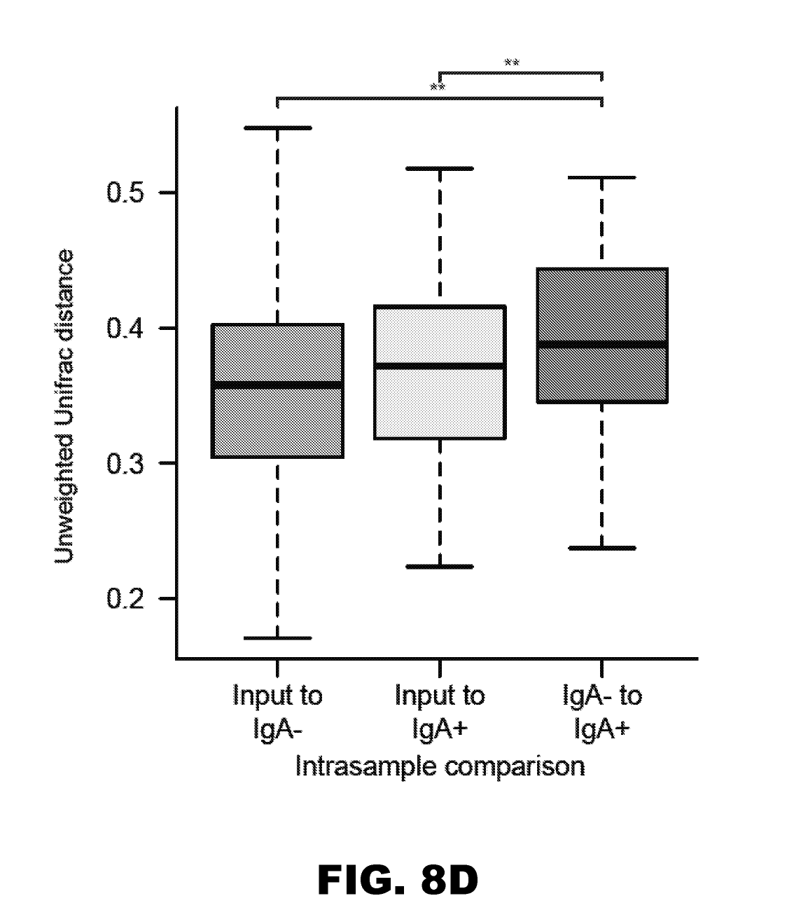

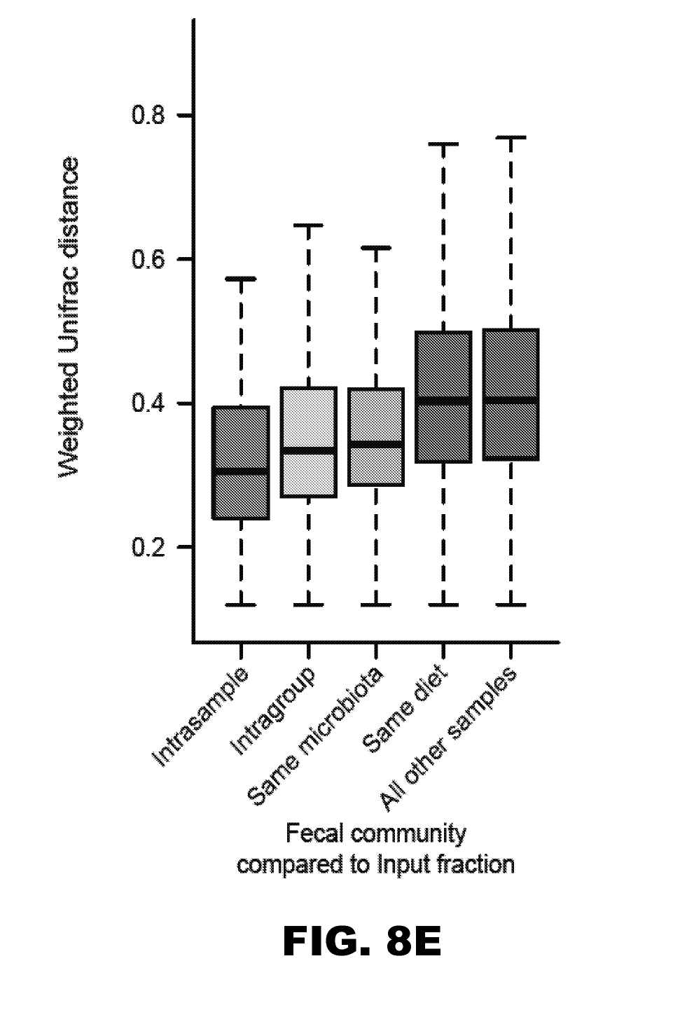

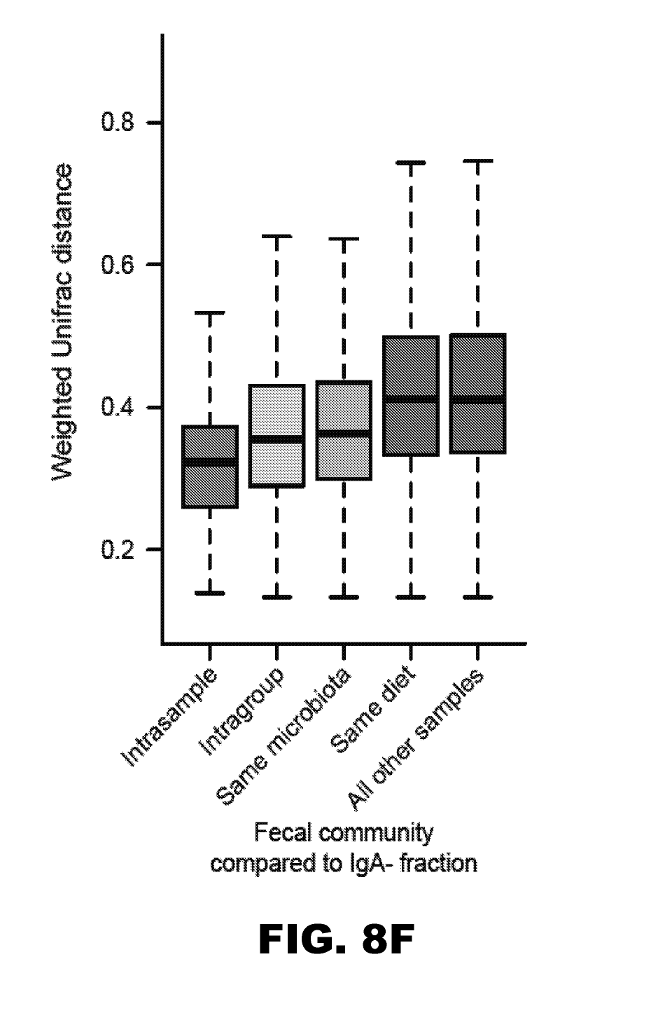

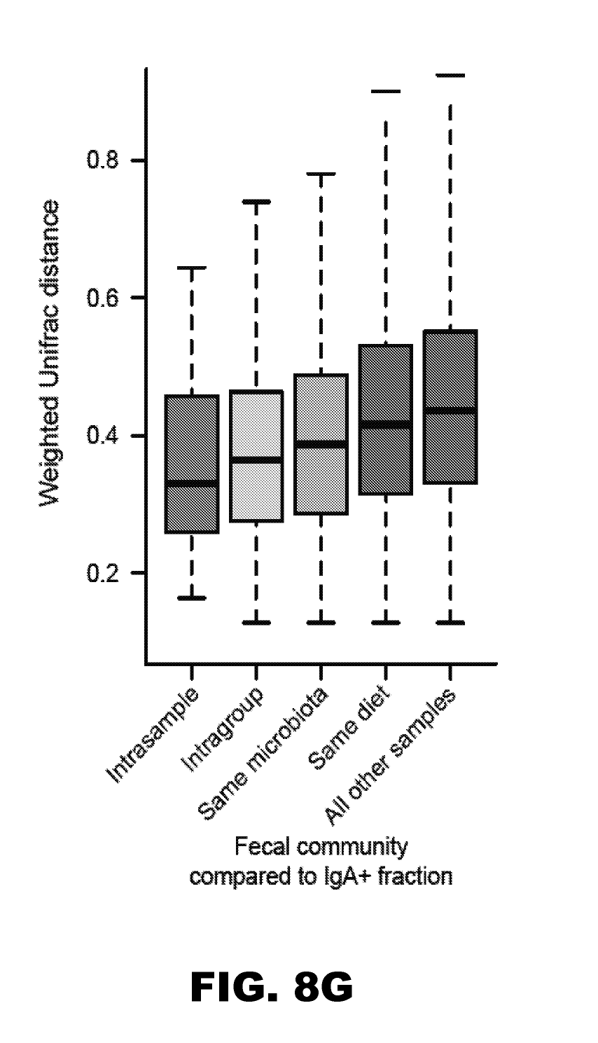

It was hypothesized that there would be a subset of the KM microbiota that would be targeted by the immune response and that these immune targeted microbes would be responsible for the weight loss observed in KM mice. To identify such targets of the immune system, analytical BugFACS was applied to these humanized mice (FIG. 5A). Members of Enterobacteriaceae were prominently enriched in the IgA+ fraction in KM mice in two independent experiments (n=5 mice, experiment 1; n=14 in experiment 2; FIG. 7A). Mice receiving a microbiota from a healthy co-twin or mice receiving a microbiota from the twin with kwashiorkor but fed a standard diet did not develop a statistically significant response to Enterobacteriaceae, despite the presence of this taxon in all experimental groups (FIG. 8A). Instead, the most prominent IgA response in mice receiving their microbiota from a healthy co-twin was against Verrucomicrobiaceae; Akkermansia muciniphila was the only representative of this family level taxon in their fecal microbiota (FIGS. 7B, 8B). While a number of other human bacterial taxa were targeted by IgA, Enterobacteriaceae was the only taxon targeted exclusively in KM mice. Erysipelotrichaceae, a member of the Firmicutes, was a target of the IgA response only in animals fed the Malawian diet, regardless of the microbiota with which they were colonized (FIG. 7C). Additional analyses of the V2-16S rRNA data generated from BugFACS of the fecal microbiota from humanized gnotobiotic mice confirmed that the proportional representation of species differed dramatically between the IgA+ and IgA− fractions (FIG. 8C; also see panels E-F).

Assaying the Functional Effects of IgA+ Consortia in Recipient Gnotobiotic Mice—

To directly test whether bacteria targeted by an IgA response are responsible for the weight loss observed in humanized KM mice, IgA bound (“IgA+”) bacteria were isolated from fecal pellets of mice colonized with either the kwashiorkor (n=3) or healthy (n=3) microbiota, and these purified consortia were transferred into germ free mice using microbial adoptive transfer. Three separate groups of mice, all maintained on the Malawian diet starting one week before gavage with the purified IgA+ consortia, were colonized with the following IgA+ fractions: (a) KM^{IgA+} mice were each gavaged with 10⁵ events (sorted IgA+ bacteria) derived from the fecal microbiota of KM mice; (b) HM^{IgA+} mice were each gavaged with 10⁵ bacteria derived from the fecal microbiota of HM mice; (c) Mix^{IgA+} mice were gavaged with a mixture of 5×10⁴ bacteria from KM mice and 5×10⁴ bacteria from HM mice so that the total number of events was also 10⁵ per mouse (FIG. 5B-C).

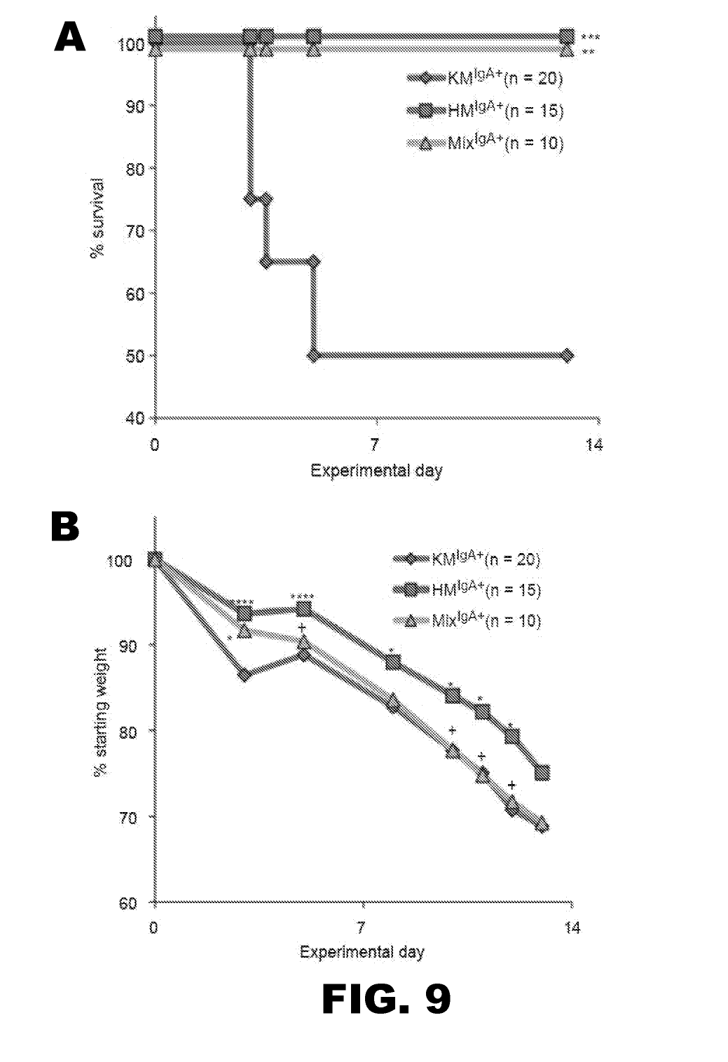

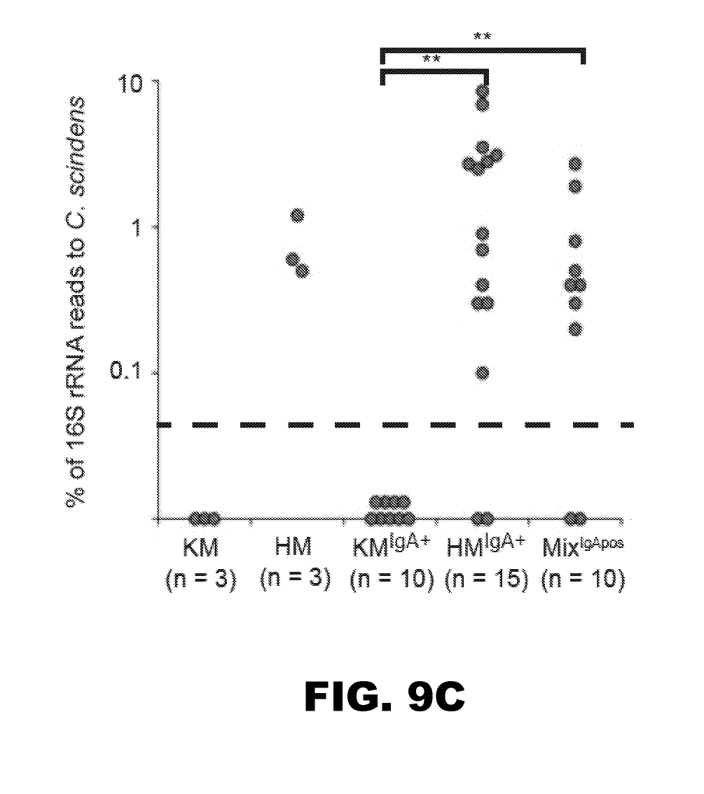

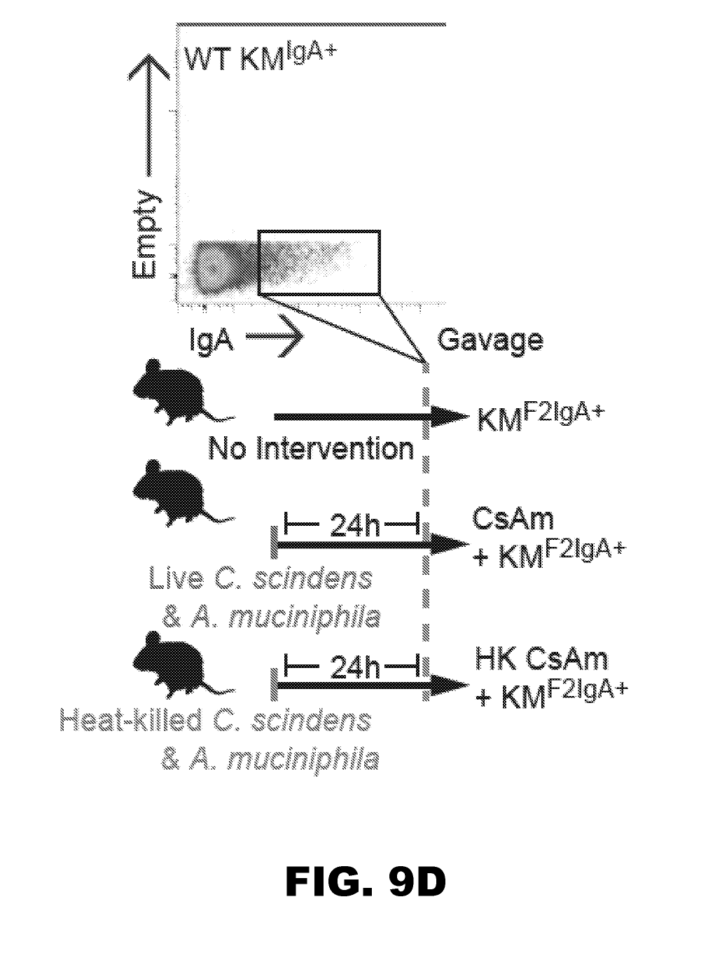

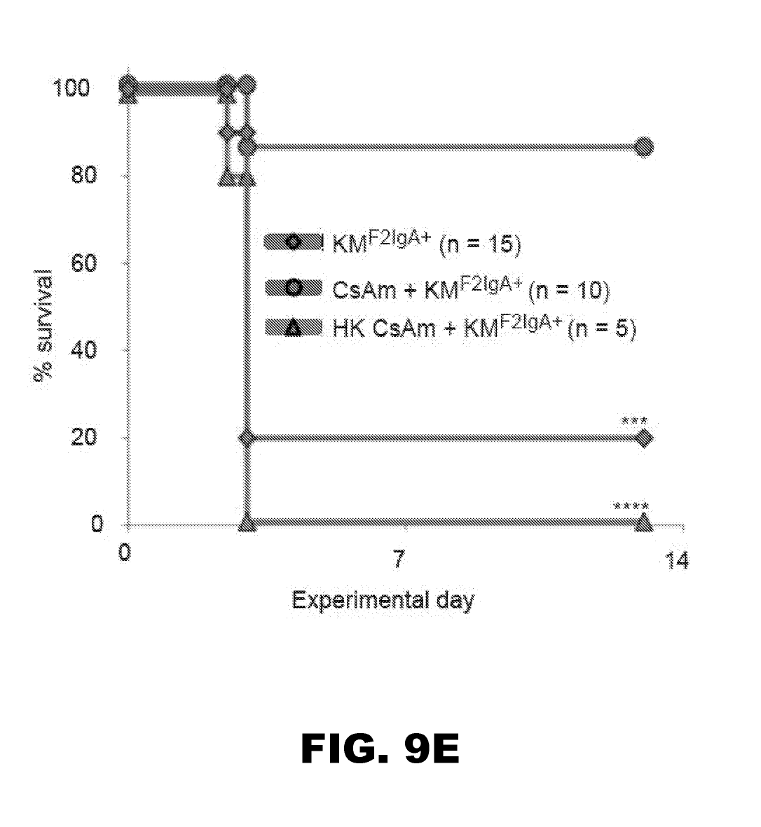

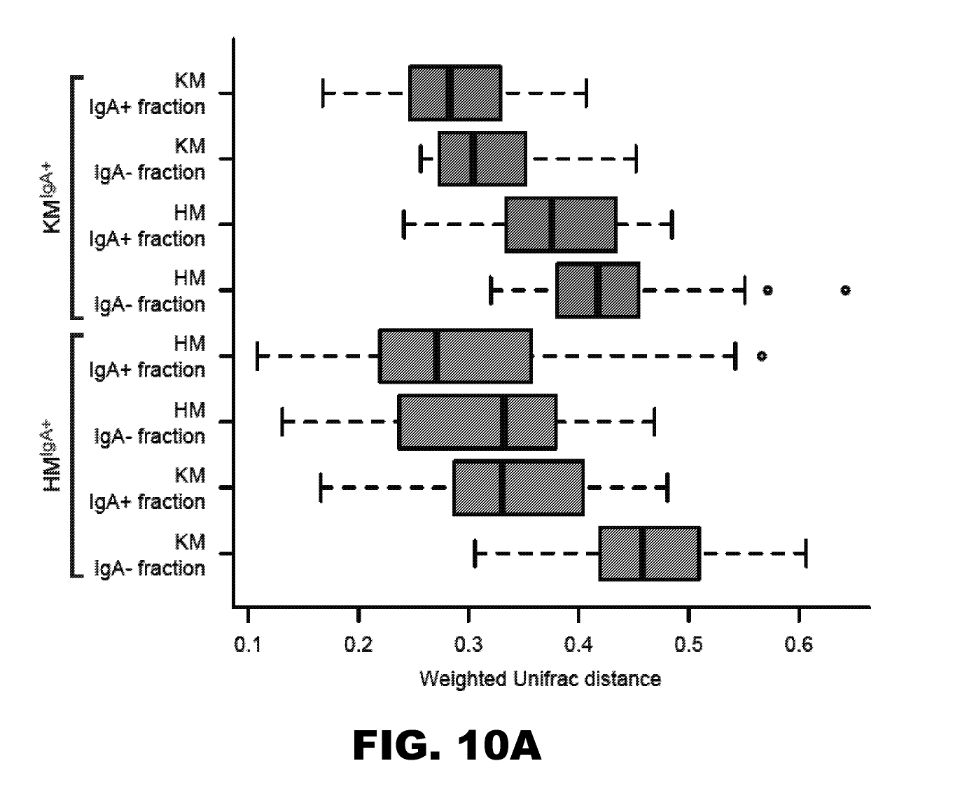

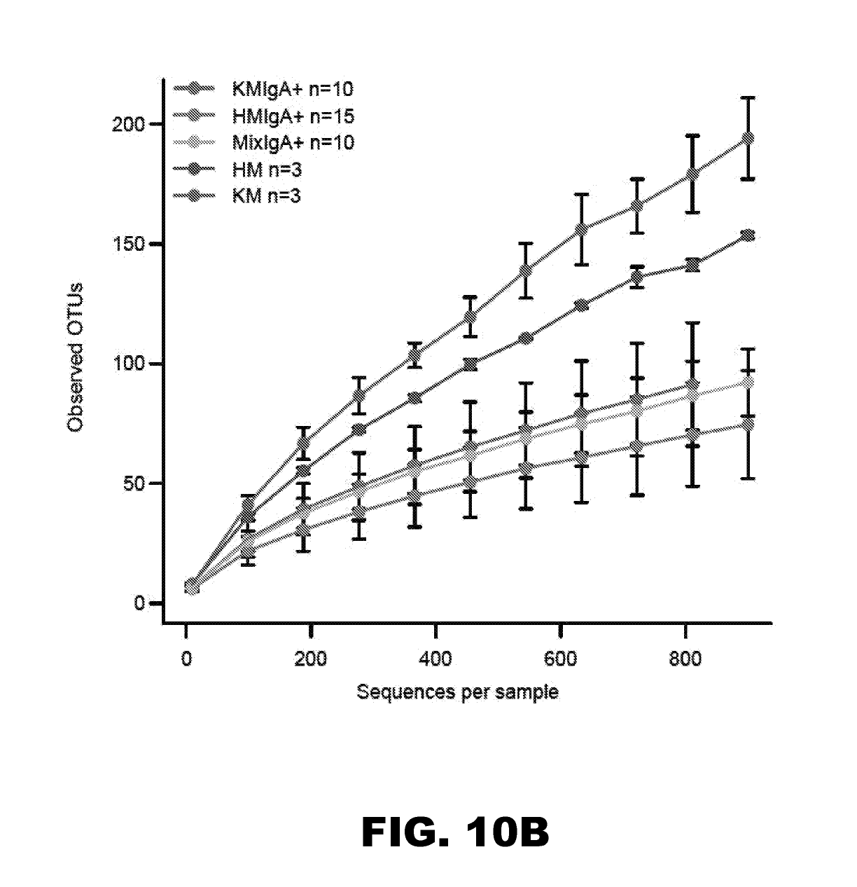

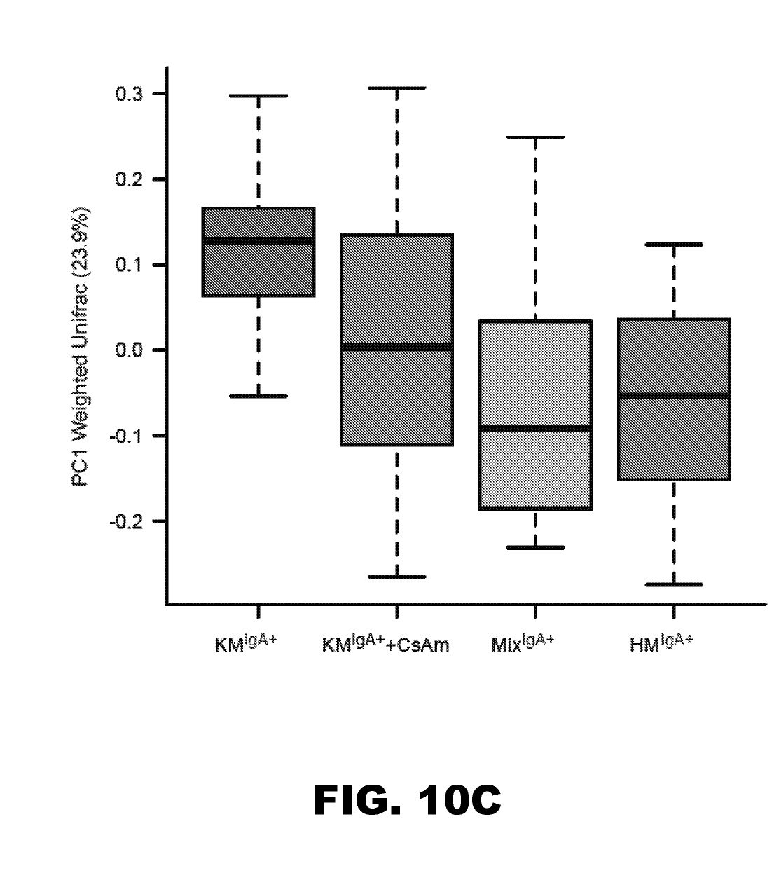

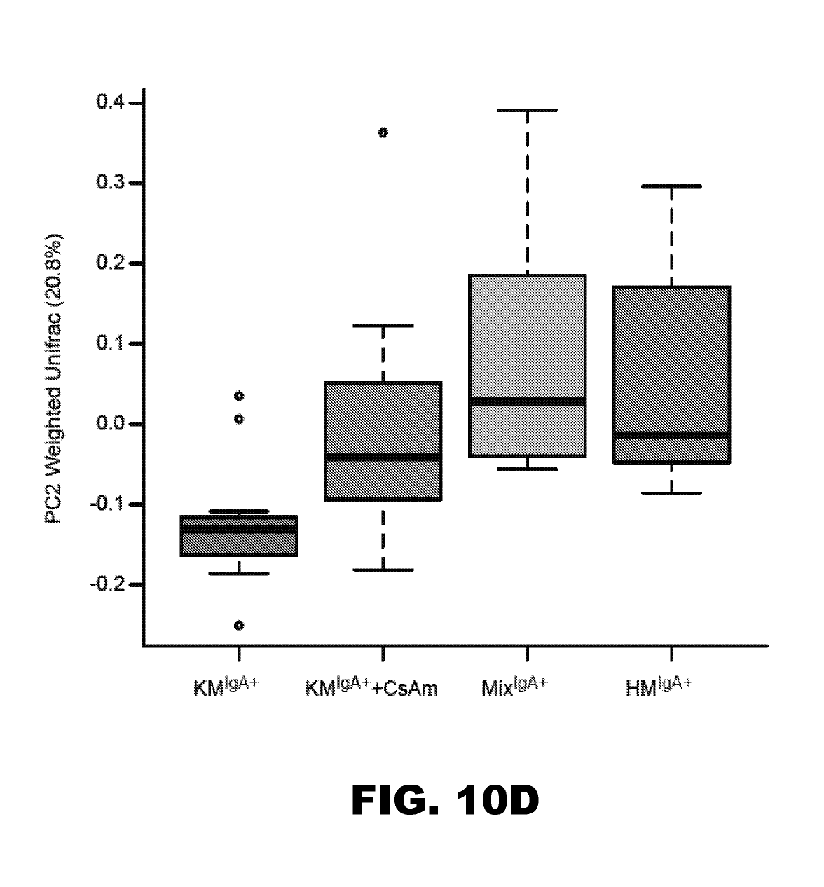

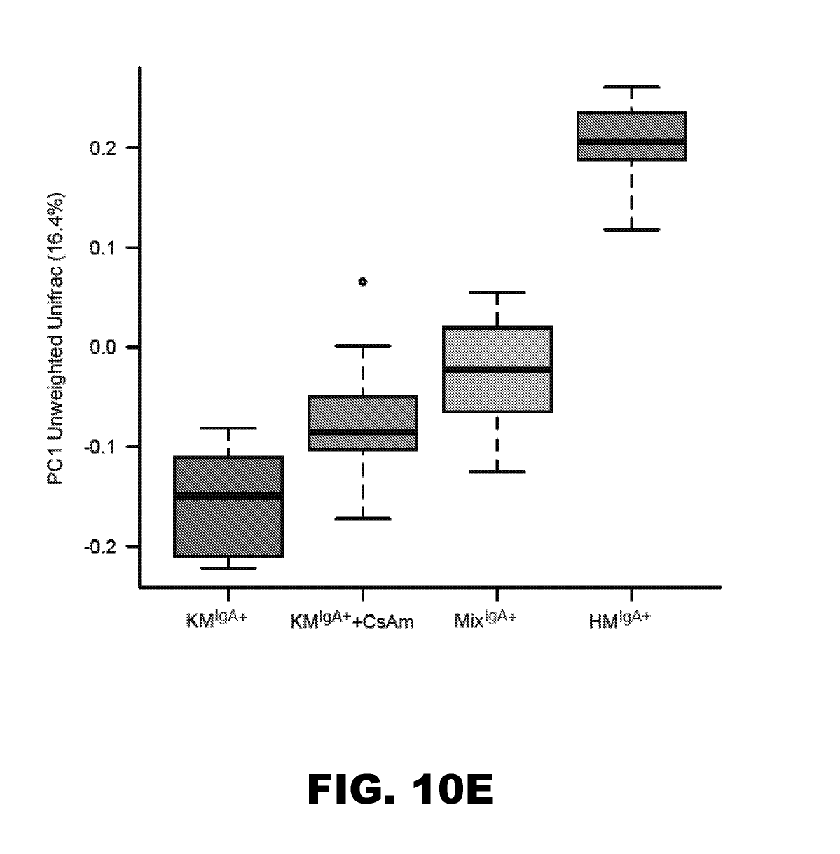

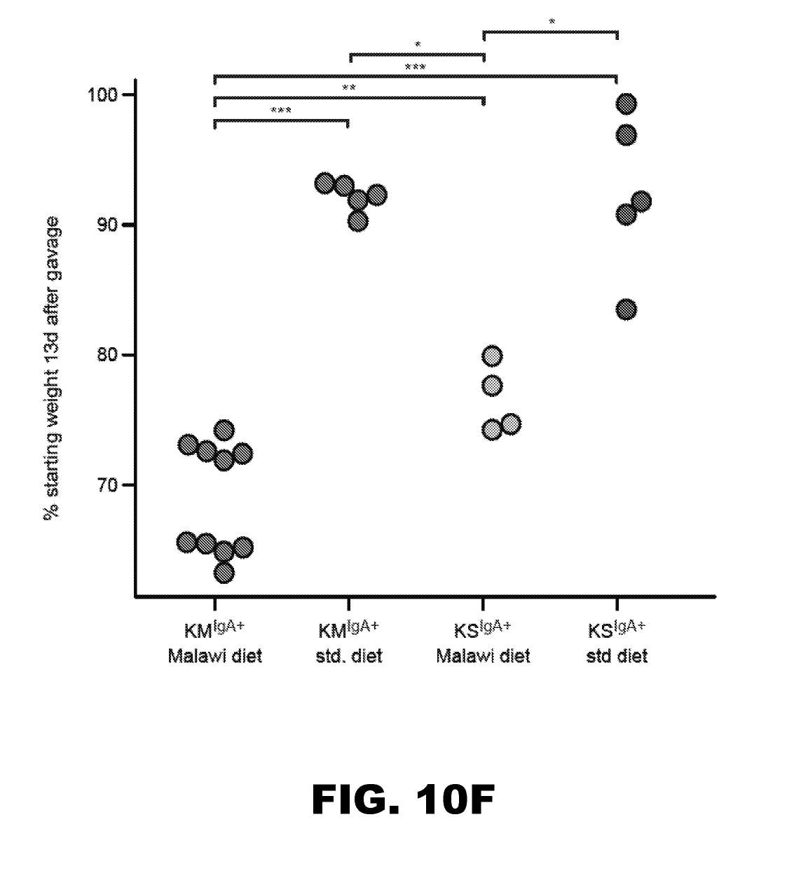

KM^{IgA+} mice fared poorly over the 13 d course of the experiment, with 50% dying within 5 d of gavage (FIG. 9A, n=20 from 2 independent experiments). In contrast, 100% of the HM^{IgA+} mice survived the full course of the experiment, despite being maintained on an identical diet and initially receiving the same total number of bacteria (n=15; 2 independent experiments). Remarkably, 100% of the Mix^{IgA+} group also survived over the entire course of the experiment, though, like the KM^{IgA+} animals, they experienced significantly more weight loss than HM^{IgA+} mice (FIG. 9B). Mortality in KM^{IgA+} mice could be prevented by feeding them a standard mouse chow rather than the Malawi diet. In addition, if the original humanized gnotobiotic mice from which the IgA+ fraction was derived had been fed a standard diet (KS mice), the recipients (KS^{IgA+} mice) experienced significantly less weight loss, and reduced mortality (FIG. 10F, n=5 mice).

Cytokine profiling of sera from moribund mice whose condition necessitated their sacrifice prior to day 13, demonstrated significant elevations in G-CSF, IL-6, IL-10, KC and IL-12p40. This cytokine signature was remarkably similar to that reported in a mouse model of cecal perforation and sepsis, suggesting barrier dysfunction leading to sepsis as the cause of death.

V2-16S rRNA sequencing of amplicons generated from the intact fecal microbiota of mice sampled 2 weeks after receiving an IgA enriched consortia helped determine differences in the community structure that could explain the differences in mortality between KM^{IgA+} and Mix^{IgA+}/HM^{IgA+} groups. Weighted UniFrac measurements revealed that the microbiota of both KM^{IgA+} and HM^{IgA+} mice most closely resembled the IgA+ fractions of the fecal communities from which they were derived. As expected, there was substantially reduced alpha-diversity in mice receiving the FACS-purified microbes when compared to humanized donor mice (FIG. 10B).

Using unweighted Unifrac, the Mix^{IgA+} communities appeared related to both the KM^{IgApos} and HM^{IgA+} microbiota (FIG. 10E). However, when using weighted UniFrac (which takes into account the relative abundance of microbes within a community), the Mix^{IgA+} mice appeared to be more similar to HM^{IgA+} mice (FIG. 10C,D). Only one species-level taxon, Clostridium scindens, satisfied our criteria of being associated with HM, HM^{IgA+} and Mix^{IgA+} mice and not KM^{IgA+} animals, suggesting a possible protective role for this bacteria (FIG. 7E).

The ability of C. scindens and A. muciniphila to prevent the mortality caused by the introduction of a purified KM^{IgA+} consortium was subsequently tested. C. scindens was selected based on its association with the microbiota of HM^{IgA+} and Mix^{IgA+} mice, and because it is related to the group of Clostridia sp. recently described to induce tolerogenic responses in mice (Atarashi et al. 2011). A. muciniphila was selected because it induced a robust IgA response in mice receiving the healthy human microbiota and because its presence has been associated with healthy, non-inflamed gut mucosa in humans (Png et al. 2010).

An equal mixture of these two taxa (abbreviated AmCs) were introduced into mice 24 h prior to introducing the IgA-enriched fraction of bacteria from KMIgA+ animals (KM^{F2-IgA+}+CsAM FIG. 9D). Control groups consisted of mice that were gavaged with a heat-killed combination of AmCs 24 h prior to introduction of the KM^{F2-IgA+} consortium (n=5 KM^{F2-IgA+}+HKCsAm) and mice that received no intervention (n=10 KM^{F2-IgA+}). In concordance with the results described in FIG. 9A, mice that received the KM^{F2-IgA+} fraction experienced a high mortality rate (˜80% within 4 d of gavage). Mice that received AmCs experienced significantly less mortality (p<0.001, chi-squared test) than control mice receiving heat-killed AmCs or no intervention.

### Example 7

### Applying BugFACS Directly to Human Fecal Samples

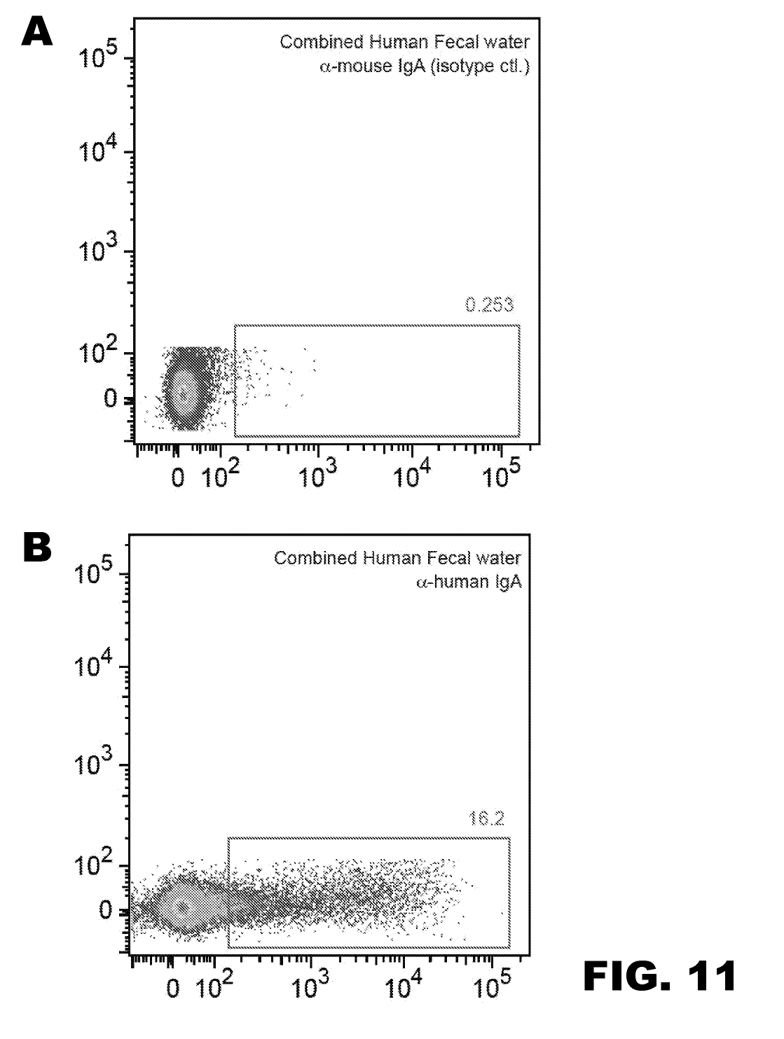

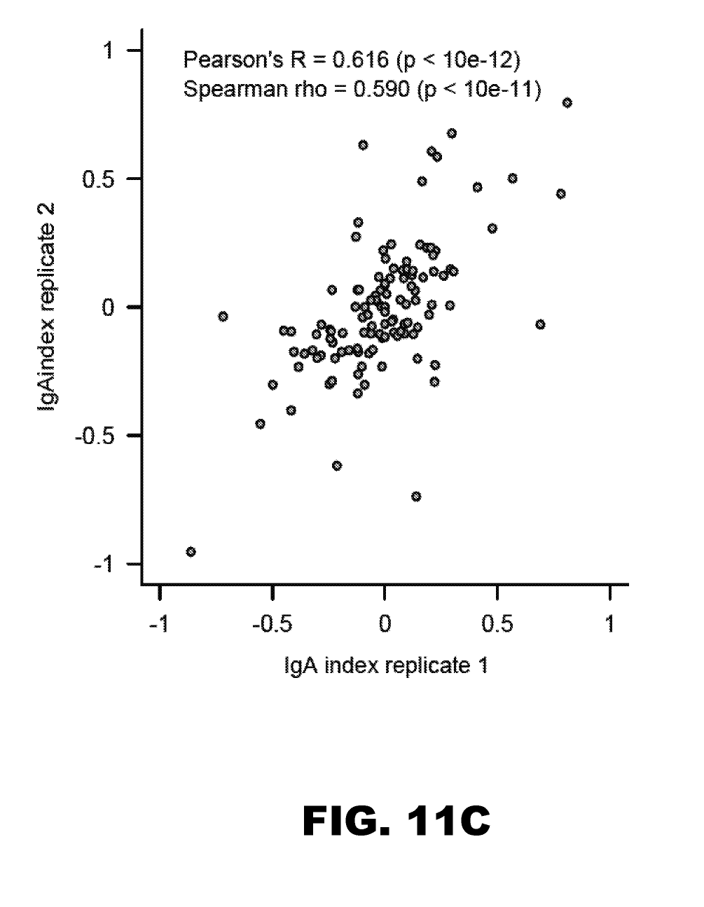

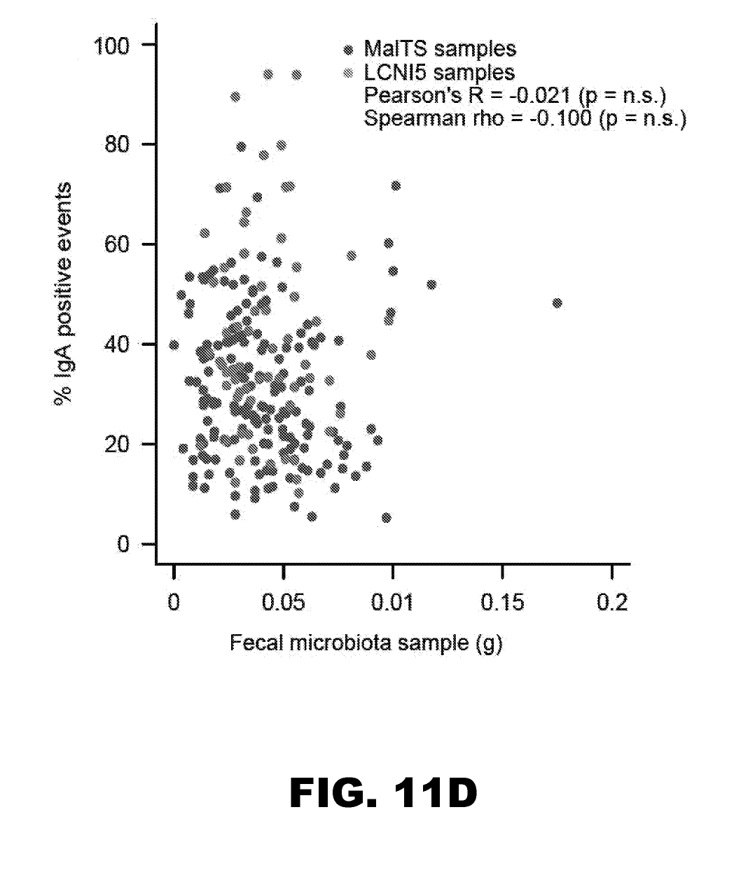

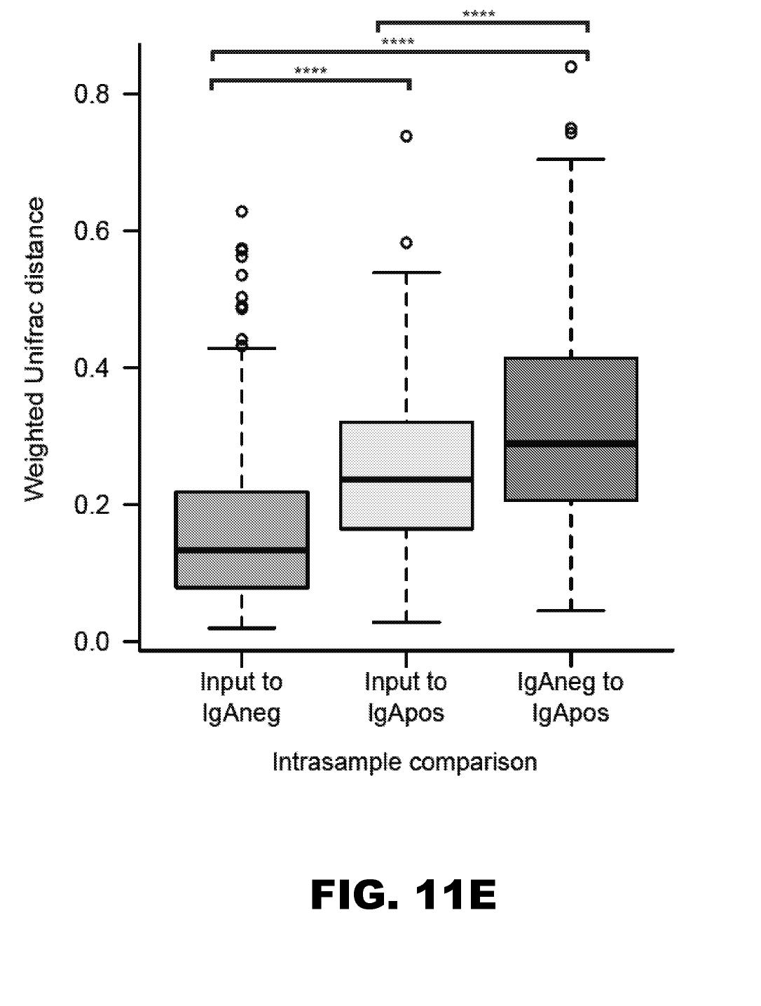

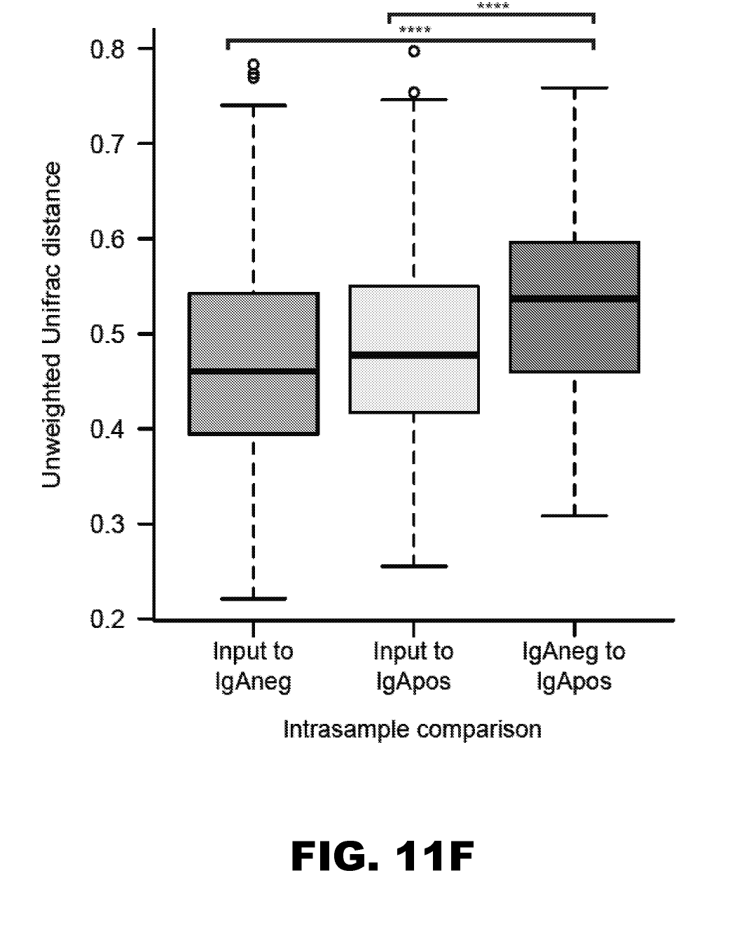

The BugFACS protocol was adapted to directly identify the bacterial targets of the human gut mucosal IgA response (rather than using fecal samples from mice that had been colonized with human microbiota) (FIG. 11 and Kau A et al, unpublished data). Using samples collected from two different clinical trials, the specificity of the anti-human IgA antibody was confirmed in our BugFACS model (FIGS. 11A, B, and D), the reproducibility of human BugFACS between replicate samples was demonstrated (FIG. 11C), and it was shown that IgA+, IgA− and input fractions maintained similarity profiles nearly identical to what was observed in humanized mice (FIG. 11E, F).

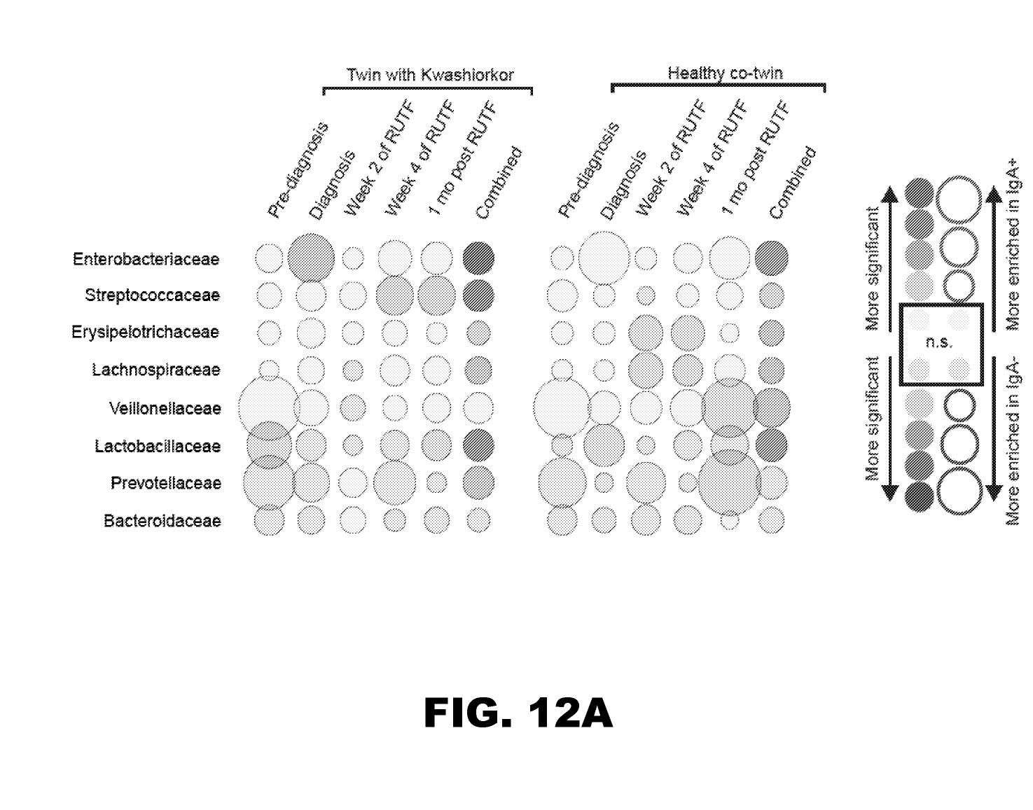

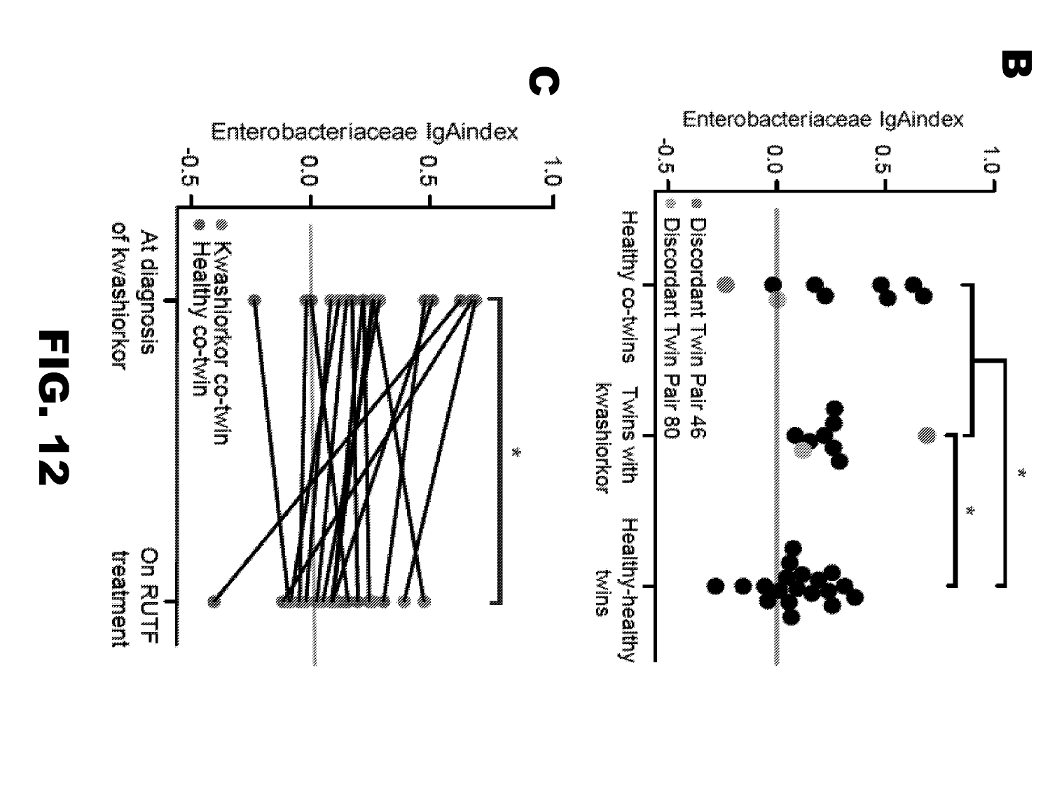

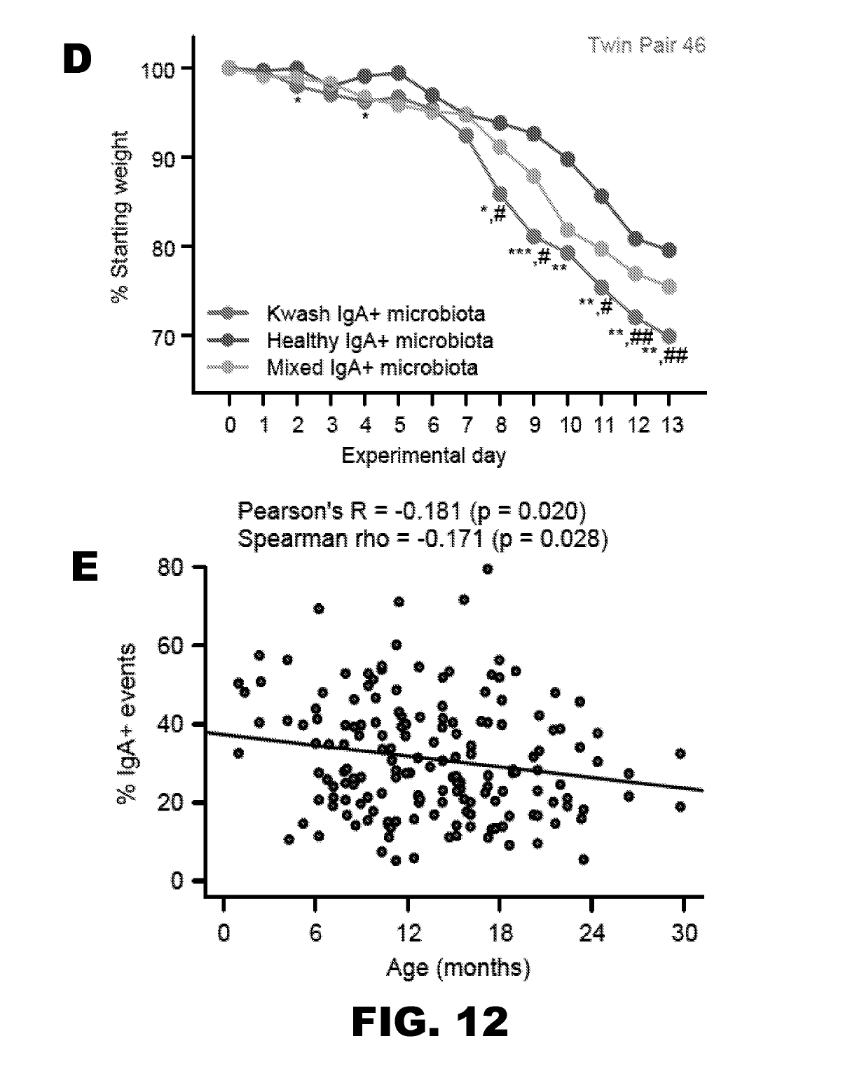

Analytical BugFACS was first applied to the Malawi twin study samples to assess to what degree the IgA responses observed in the gnotobiotic mouse studies were generalizable to human samples. Included in the analysis were 11 twin pairs discordant for kwashiorkor as well as 15 healthy-healthy twin pairs that were included as controls. Samples obtained from twins with kwashiorkor at the time of diagnosis demonstrated significant IgA targeting of Enterobacteriaceae (FIG. 12A). The healthy co-twins of these children with kwashiorkor showed much more variable targeting of Enterobacteriaceae by IgA. When compared to the IgA responses seen in Healthy-Healthy co-twin pairs, Enterobacteriaceae was targeted to a significantly greater degree in discordant twin pairs (FIG. 12B). During RUTF treatment, the IgA response to Enterobacteriaceae decreased in both kwashiorkor and healthy co-twins (FIG. 12C).

To directly assess the role of IgA bound microbes from discordant twins, two twin pairs were selected based on the strength of their IgA targeting of Enterobacteriaceae to perform microbial adaptive transfer into germ-free recipient mice. In twin pair 46, it was observed that the degree of IgA targeting of Enterobacteriaceae was much higher in the kwashiorkor compared to healthy co-twin. Twin pair 80 showed only a small difference in the degree of IgA targeting between the kwashiorkor and healthy siblings. For each twin pair, the IgA+ microbes from the healthy co-twin, the co-twin with kwashiorkor and a “mixed” microbiota consisting of a 1:1 mixture of the healthy and kwashiorkor IgA+ microbes were transplanted into germ free animals (n=6-7 animals/group) fed a Malawi diet. Mice receiving the IgA+ kwashiorkor co-twin's microbiota from twin pair 46 demonstrated a significantly greater degree of weight loss when compared to mice receiving the healthy co-twin's IgA+ consortium or the IgA+ mix (FIG. 12D). Healthy IgA+, kwashiorkor IgA+ or Mix IgA+ fractions from twin pair 80 did not produce significant differences in their effects in gnotobiotic recipients.

Evolution of IgA Responses in Discordant Twin Pairs and in Twin Pairs Concordant for Healthy Status—

BugFACS analysis of fecal samples revealed that responses against Bifidobacteriaceae increased as a function of the age regardless of the health status of the child, suggesting at least some ordered ontogeny of IgA responses. Consistent with this idea, the absolute fraction of IgA+ events decreased with age (FIG. 12E), which may suggest a gradual shift from broadly specific responses early in life to more specific IgA responses later in childhood.

### REFERENCES

1. S. Kawamoto et al., The Inhibitory Receptor PD-1 Regulates IgA Selection and Bacterial Composition in the Gut, Science 336, 485-489 (2012).
2. S. Hapfelmeier et al., Reversible Microbial Colonization of Germ-Free Mice Reveals the Dynamics of IgA Immune Responses, Science 328, 1705-1709 (2010).
3. C. F. Maurice, H. J. Haiser, P. J. Turnbaugh, Xenobiotics shape the physiology and gene expression of the active human gut microbiome, Cell 152, 39-50 (2013).
4. D. A. Peterson, N. P. McNulty, J. L. Guruge, J. I. Gordon, IgA Response to Symbiotic Bacteria as a Mediator of Gut Homeostasis, Cell Host and Microbe 2, 328-339 (2007).
5. M. I. Smith et al., Gut microbiomes of Malawian twin pairs discordant for kwashiorkor, Science 339, 548-554 (2013).
6. K. Atarashi et al., Induction of colonic regulatory T cells by indigenous Clostridium species, Science 331, 337-341 (2011).
7. C. W. Png et al., Mucolytic bacteria with increased prevalence in IBD mucosa augment in vitro utilization of mucin by other bacteria, Am. J. Gastroenterol. 105, 2420-2428 (2010).
8. A. L. Goodman et al., Extensive personal human gut microbiota culture collections characterized and manipulated in gnotobiotic mice, Proc. Natl. Acad. Sci. U.S.A. 108, 6252-6257 (2011).

### Example 8

### Discussion of Examples 5-7

Given (i) the importance of mucosal barrier function to health, (ii) the role of mucosal barrier dysfunction in disease, notably diseases involving a breakdown of the normal homeostasis that exists between indigenous microbial communities occupying various body habitats and the immune system, (iii) the difficulty in directly accessing mucosal surfaces in remote parts of the bodies of humans or veterinary animals (e.g. along the length of the gastrointestinal tract), and (iv) the intrapersonal and interpersonal variations that exist in microbial community structure and function, there is a need for new inventive methods that allow viable organisms that are recognized or ignored by the mucosal immune system to be collected and manipulated. The new methods create a new way for characterizing mucosal barrier/immune function within an individual as a function of their age, physiologic/health status and environmental exposures, and between groups of individuals as a function of their age/health status and environmental exposures. They also provide a way for identifying new agents that can protect, repair or fortify mucosal barrier function and health.

This novel approach of identifying microorganisms that are targets of an immune response may prove to be an effective means of identifying microorganisms that convey a particular host phenotype. Generation of an IgA response to a particular organism is probably most closely correlated to the biogeography of that organism-organisms that develop a close association with the mucosal barrier are most likely to be targeted by IgA. In non-pathologic states, bacteria that are targets of an IgA response are probably those bacteria best adapted to survive close to the mucosal surface. In pathologic conditions, disease-causing microbes may displace the normal mucosal associated microbiota, and by doing so, render the host tissue more susceptible to damage. Data presented here suggest that the factors that can lead to a change in the targets of an IgA response will include not only microbial exposures, but also diet changes provoking changes in the microbial community structure and/or the surface (antigenic) features of community members.

The ability to identify consortia of bacteria known by the immune system at any given point in time (‘immunognostic’ organisms), may have important prognostic implications, particularly in conditions where mucosa-associated bacteria play important roles in host barrier as well as microbial community functions. These data support a key role for Enterobacteriaceae in exacerbating and predisposing children to severe malnutrition. The role of diet in shaping these immune responses is particularly important, as effective dietary intervention may be most effective when it provides vital nutrients and changes the mucosal microbial community to modulate the mucosal immune response.

Immunognostic organisms may also be of particular interest in the formulation of tailored probiotics. These data suggest that not all bacterial targets of IgA are detrimental to the host and may, in some cases, be indicative of a salubrious microbiota-host interaction. Efforts to identify bacteria that are frequently targets of the host IgA response in diseased and healthy states should aid our understanding of microbiota/immune interactions and help identify potential therapeutic organisms.

### Example 9

### Materials and Methods for Examples 5-7

Malawi Twin Study—

This clinical trial has been described in a prior publication (Smith_Yatsunenko et al., 2013). Briefly, twins were recruited to the study at one of five sites in Malawi: Makhwira, Mitondo, M'biza, Chamba, and Mayaka. A team of American and local personnel visited each site on a monthly basis, measured height and weight, and screened children for pitting edema of the lower extremities. Fecal specimens were collected every three months for twins that remained concordant and healthy. In twin pairs where one twin developed kwashiorkor, both twins were switched to a peanut-based Ready to Use Therapeutic Food (RUTF). Sampling of fecal specimens was increased to bi-weekly while children were receiving RUTF. Fecal specimens were flash frozen in liquid nitrogen and stored at −80 C prior to analysis. Human study protocols were approved by the Human Research Protection Office of Washington University School of Medicine and the College of Medicine Research Ethics Committee of the University of Malawi.

Food Preparation of Diets for Gnotobiotic Mouse Studies—

The Malawian diet was based on food consumption patterns in the catchment area, and consisted primarily of corn flour, mustard greens, yellow onions and tomatoes purchased from a US based vendor. Batches of diet were aliquoted into 500 g or 750 g vacuum pouches, placed in second exterior bag and sterilized by irradiation. Sterility was confirmed using standard culture methods. Aliquots of food aliquots were stored for up to 6 months at 4° C. Nutritional analysis was performed by N.P. Analytical labs.

Sorting of IgA Coated Bacteria from Fecal Specimens

Fecal Specimen Preparation—

Whole mouse fecal pellets, weighing ˜10-50 mg were suspended in 1 mL of sterile PBS by vigorous vortexing for 5 min. Samples were then placed on ice and allowed to undergo gravity sedimentation for 5-10 min. 200 μL of the clarified fecal suspension was then passed through a 70 micron-diameter sterile nylon mesh filter into a new, sterile tube. Filtered bacteria were then pelleted by centrifugation. The cell-free supernatant was removed and the pellet washed by resuspension in an additional 1 mL of PBS and again centrifuged. The resulting pellet was resuspended in 100 μL of PBS containing a 1:50 dilution of polyclonal goat anti-mouse IgA antibody conjugated to DyLight649 (Abcam) and incubated at 4° C. After 30 min, the suspension was washed with 1 mL of sterile PBS and pelleted again by centrifugation. We then added 200 μL of 0.9% NaCl and 0.1 M HEPES buffer containing a 1:4000 dilution of SytoBC (Invitrogen/Life Sciences).

Human fecal samples (20-100 mg) were processed as above and stained with a goat anti-human IgA antibody conjugated to DyLight649 (Abcam). Both pulverized and non-pulverized samples were utilized for these studies.

Sorting of Bacteria—

A FACS Aria III (BD Biosciences) instrument was used to sort bacteria. Sheath fluid (PBS) was sterilized by autoclave immediately before use. Flow cytometers were sterilized according to the manufacturer's recommended protocol, prior to sorting. Contamination of the cytometers was monitored by V2 16S PCR of sheath fluid flow through before and after bacterial sorting. We prevented exposure to aerosolized fecal samples by sorting human samples strictly within a laminar flow bio-containment hood. Fecal samples were analyzed without the use of a neutral density filter to allow the maximum degree of sensitivity for small particles. Threshold settings were set to the minimal allowable voltage for SSC. The gating strategies used to collect different bacterial populations are shown in FIG. 1. We collected 20,000-50,000 events from the IgA+ population and a minimum of 100,000 events from the IgA− and ‘All’ populations into sterile Eppendorf tubes. All sorted bacteria were stored at −80° C. prior to 16S rRNA PCR

V2 16S rRNA PCR—

Crude DNA was prepared from fecal samples by bead beating, followed by phenol-chloroform extraction and amplified using barcoded V2 16S rRNA primers. PCR was performed using either 5prime HotMaster Mix or Invitrogen High Fidelity Platinum Taq according to the manufacturer's protocols.

To amplify V2 16S rRNA sequences from sorted bacteria, we added 1 μL of the purified fractions to PCR mastermix (three replicate 20 μL reactions). Cycling conditions were as follows: 95° C. for 10 min; followed by 30 or 35 cycles of 95° C. for 20 s, 52° C. for 20 s and 65° C. for 60 s; then 4° C. ‘Noεtmplate’ controls were run with every sample to ensure that there was no contamination of primers or reagents. The products of PCR reactions were subjected to gel electrophoresis (to confirm the presence of amplicon products), quantified and pooled. Multiplex V2-16S rRNA amplicon sequencing was performed with a 454 Pyrosequencer using Titanium FLX chemistry.

Analytical Pipeline—

We de-multiplexed and clustered V2-16S 454 operational taxonomic units (OTUs) at 97% identity using the uclust method in QIIME version 1.4. Data were filtered so that each sample had at least 1000 reads and each OTU had to be observed at lease twice across all samples. We assigned taxonomy to OTUs using RDP 2.4 trained on a custom database derived from sequence data downloaded from the Greengenes ‘Named Isolates’ database and phylogeny assigned from the NIH's database. OTUs were rarefied to an even depth of 1000 reads per sample prior to analysis.

To identify taxa that were targets of an IgA response, we summarized taxonomy to species, genus, family, order, class and phylum levels with the output given in proportional representation of each taxa. A paired t-test was applied, comparing the IgA+ to IgA− populations within a group of samples. A pseudocount (equivalent to a single read, or 0.001) was added to every sample and the data were log transformed before performing the paired t-test using Perl and/or R. (Differences between IgA+ and IgA− populations followed a log normal distribution.) The IgA index was calculated as outlined in the text with pseudocounts included.

### Gnotobiotic Mouse Experiments

Adult (7-12 week old) germ-free male C57 BL/6J mice were maintained on a 12 h light cycle (lights on at 0600) in flexible plastic isolators (Class Biologically Clean Ltd., Madison, Wis.) as previously described. Mice were weaned onto an autoclaved nutritionally replete chow (B&K Universal, East Yorkshire, U.K; diet 7378000) until 7d before introduction of the gut microbiota, when some animals were switched to the Malawi diet, as described in the text. The Washington University Animal Studies Committee approved all animal study protocols described in this paper.

Humanized Gnotobiotic Animals—

Clarified suspensions were prepared in reduced PBS from fecal samples obtained from a single discordant twin pair (twin pair 57) at the time of diagnosis of kwashiorkor in one of the co-twins. A 200 μL aliquot of this suspension was introduced into germ-free mice by gavage (Smith/Yatsunenko et al., 2013). As a physiologic measure of mucosal immune function, we vaccinated all humanized mice with oral cholera toxin and ovalbumin starting at day 21 post-gavage (days 21, 28 and 35 for group 1; only day 21 for group 2). Each dose of vaccine contained 10 μg of cholera toxin and 10 mg of hen egg ovalbumin (Sigma, St. Louis, Mo.) dissolved in sodium bicarbonate pH 8.0. Vaccines were mixed and filter sterilized (0.22 micron-diameter filter) prior to their administration by gavage.

Sorting of Enriched IgA Population for Transplantation to Gnotobiotic Mice—

All preparation and staining steps took place within an anaerobic chamber (Coy Lab Products, Grass Lake, Mich.; atmosphere composed of 75% N₂/20% CO₂/5% H₂). All buffers used during the preparation and staining steps (PBS and 0.1 M HEPES/0.9% NaCl) were supplemented with 0.1% cysteine HCl and stored buffers anaerobically for a minimum of 24 hours before usage. Plasticware used for preparing samples were also stored anaerobically for a minimum of 3 days prior to use. For steps requiring centrifugation, we used sealed tubes (Axygen 2 ml screwtop tubes) to centrifuge samples outside of the anaerobic environment and returned samples to the anaerobic atmosphere prior to additional processing.

Fecal pellets used to generate KM^{IgApos}, HM^{IgApos} and Mix^{IgApos} mice came from humanized KM and HM mice (group 1 animals, see FIG. 4) 42 days after introduction of the human fecal microbiota. We combined the filtered fecal suspensions of each of the three surviving mice in each of the KM and HM groups to generate a pooled microbiota that was subsequently stained and used to extract IgA-enriched bacteria. Similarly, KM^{F2-IgApos} were generated from the combined, filtered fecal supernatants from five surviving KM^{IgApos}. (KM^{F2-IgApos} animals are the third generation of mice harboring a microbiota derived from one of the Malawian co-twins and the second generation of mice receiving an IgA enriched microbiota.)

Bacteria were sorted under normal (non-anerobic) conditions using SSC or FSC and SSC at the minimum possible voltages as threshholds. In order to minimize oxygen exposure of specimens during sorting, we periodically retrieved fresh aliquots of stained fecal specimens from the anaerobic chamber during the sorting process. Additionally, bacteria were sorted into 2 ml of reduced PBS 0.1% cysteine. Once a sufficient number of events were collected, sorted bacteria were centrifuged, the supernatant was removed and the pelleted microbes resuspended in a volume of PBS/0.1% cysteine sufficient to deliver 100,000 events in 200 μL. Sorted bacteria were sealed in the anaerobic chamber and immediately transferred into gnotobiotic isolators to be gavaged into germ-free or probiotic treated animals.

Probiotic Intervention—

Akkermansia muciniphila ATCC BAA-835 and Clostridium scindens ATCC 35704 were obtained from ATCC (Manassas, Va.). Both strains were grown overnight at 37° C. in Gut Microbbta Medium (Goodman et al., 2011) under strict anaerobic conditions. Equivalent numbers of organisms in the two cultures were mixed (normalizing to OD600), the bacteria were pelleted by centrifugation and resuspended in PBS/0.1% cysteine so that the final OD600 was 1. Precautions were taken to limit exposure of probiotic consortia to oxygen by conducting manipulations within a Coy chamber and sealing tubes with parafilm when samples had to be transported (e.g. during centrifugation and at the time of gavage).

1. A method for identifying a physiological state of a subject, the method comprising
a) combining one or more biological samples comprising an immune system: microorganism complex obtained from the subject with one or more detection agents;
b) sorting, in vitro, the one or more samples into two populations: a detection agent bound immune system: microorganism complex population and an unbound immune system: microorganism complex population;
c) identifying the taxonomic composition of one or more detection agent bound immune system: microorganism complex populations and identifying the taxonomic composition of one or more unbound immune system: microorganism complex populations from the one or more samples;
d) calculating a strength of enrichment for an identified taxon in the detection agent bound population compared to the unbound population from each sample, wherein a strength of enrichment value greater than zero indicates enrichment in the detection agent bound population; and
e) identifying the physiological state of the subject by comparing the taxa enriched in the detection agent bound population of the subject to one or more reference samples each associated with a physiological state, wherein if the enriched taxa are similar between the subject and the reference sample, the subject has the physiological state associated with the reference sample.

2. The method of claim 1, wherein the detection agent is specific for an immunoglobulin (Ig) selected from the group consisting of IgG, IgM, IgE, IgA, IgD, and mixtures thereof, and wherein the strength of enrichment is selected from the group consisting of the IgG index, the IgM index, the IgE index, the IgA index, and the IgD index.

3. (canceled)

4. The method of claim 1, wherein the biological sample is obtained from a mucosal surface selected from the group consisting of gastrointestinal, genitourinary, oral, nasopharyngeal, vaginal, pulmonary, skin, eye, sinus, or combinations thereof.

5. The method of any of claim 1, wherein the detection agent is an antibody and in step (b) the microorganisms are sorted using a method selected from the group consisting of fluorescence activated cell sorting (FACS), immunoprecipitation, or antibody-bead conjugated separation, or combinations thereof.

6. (canceled)

7. The method of claim 1, wherein the taxonomic composition is identified at a level selected from the group consisting of species, genus, family, order, class, phylum and a combination thereof.

8. The method of claim 1, wherein the physiological state is proper functioning of the mucosal barrier including its immune cell population, and the disruption of that function, as for example in the case of forms of malnutrition, where the subject is a mammal, the biological sample is a fecal sample, and the detection agent is an anti-IgA antibody.

9. The method of claim 1, wherein an enrichment of Enterobacteriaceae indicates malnutrition or an increased risk of malnutrition.

10. The method of claim 1, wherein the microorganisms present in the detection agent bound population and unbound population are viable.

11.-12. (canceled)

13. A method of identifying one or more taxa targeted by the immune system of a subject comprising:
a) mixing a biological sample comprising microorganisms from different taxa from the subject with one or more detection agents;
b) sorting the sample into two populations: a detection agent bound microorganism population and an unbound microorganism population;
c) identifying the taxonomic composition of the detection agent bound microorganism population and the unbound microorganism population;
d) comparing the taxonomic composition of the detection agent bound microorganism population to the unbound microorganism population; and
e) calculating a strength of enrichment for each taxon in the detection agent bound population;
wherein a strength of enrichment value greater than zero indicates enrichment of the identified taxa in the detection agent bound population and targeting by the immune system.

14. The method of claim 13, wherein the biological sample comprises at least one immunoglobulin: microorganism complex, and, wherein the detection agent is specific for an immunoglobulin (Ig) selected from the group consisting of IgG, IgM, IgE, IgA, IgD, and mixtures thereof, and wherein the strength of enrichment is selected from the group consisting of the IgG index, the IgM index, the IgE index, the IgA index, and the IgD index.

15.-16. (canceled)

17. The method of claim 13, wherein the detection agent is an antibody and in step (b) the microorganisms are sorted using a method selected from the group consisting of fluorescence activated cell sorting (FACS), immunoprecipitation, or antibody-bead conjugated separation, or combinations thereof.

18.-19. (canceled)

20. The method of claim 13, wherein the biological sample is a fecal sample and the detection agent is an anti-IgA antibody.

21. The method of claim 13, further comprising culturing the detection agent bound microorganism population, wherein the method of culturing is selected from the group consisting of (i) inoculating the detection agent bound microorganism population into a germ free animal, (ii) growing the detection agent bound microorganism population in vitro using standard anaerobic techniques, and (iii) a combination thereof.

22. The method of claim 13, wherein the microorganisms present in the detection agent bound population are viable.

23.-24. (canceled)

25. A method of screening for a therapeutic intervention effective at modulating the immune response, the method comprising:
a) administering to one or more subjects one or more therapeutic interventions, wherein
i) the subject is a model of a physiologic state and the taxa targeted by the immune system in the subject are known, and
b) identifying one or more taxa targeted by the immune system of the subject after administration of the therapeutic intervention to the subject, wherein the one or more taxa targeted by the immune system are identified by the method of claim 13; and
c) comparing the strength of enrichment for each taxon in the detection agent bound population before and after administration of the therapeutic intervention to the subject;
wherein a change in the strength of enrichment after administration as compared to before administration of the therapeutic intervention indicates the therapeutic intervention was effective at modulating the immune response.

26. The method of claim 25, wherein the therapeutic intervention is selected from the group consisting of a compound, a biologic, a probioitic, a prebiotic, a synbiotic, an antibiotic, a change in diet, and a combination thereof.

27. The method of claim 25, wherein the therapeutic intervention is a composition comprising Clostridium scindens, Akkermansia muciniphila, or a combination thereof.

28. The method of claim 25, wherein the change is a decrease in the enrichment of Enterobacteriaceae.

29. The method of claim 25, wherein the change is an increase in the enrichment of Clostridium scindens, Akkermansia muciniphila, or a combination thereof.

30.-57. (canceled)
